(19) United States
(12) Patent Application Publication
(10) Pub. No.: US 2024/0245721 A1
(43) Pub. Date: Jul. 25, 2024

(54) DOUBLE SIDED CHIMERIC ANTIGEN RECEPTOR (CAR) ENGINEERED CELL MEMBRANE BASED DRUG DELIVERY SYSTEMS

(71) Applicant: BOARD OF REGENTS, THE UNIVERSITY OF TEXAS SYSTEM, Austin, TX (US)

(72) Inventors: Kytai NGUYEN, Austin, TX (US); Jon WEIDANZ, Austin, TX (US); Serkan YAMAN, Austin, TX (US); Harish RAMACHANDRAMOORTHY, Arlington, TX (US); Manoj Kumar SABNANI, Arlington, TX (US)

(21) Appl. No.: 18/558,882

(22) PCT Filed: May 5, 2022
(86) PCT No.: PCT/US2022/027883

§ 371 Date: Nov. 3, 2023

Related U.S. Application Data
(60) Provisional application No. 63/184,769, filed on May 5, 2021.

(51) Int. Cl. A61K 35/17; A61K 9/16; A61K 39/00; C07K 14/725; C07K 16/32

(52) U.S. Cl. CPC A61K 35/17; A61K 9/1647; A61K 39/4611; A61K 39/4631; A61K 39/464406; C07K 14/7051; C07K 16/32; A61K 2239/13; A61K 2239/29; A61K 2239/49; A61K 2239/55; C07K 2317/31; C07K 2317/53; C07K 2317/622; C07K 2319/03

(57) ABSTRACT

Disclosed are chimeric antigen receptor (CAR) engineered T lymphocyte membrane coated nanoparticles (CAR-T MNP) compositions and methods of using the same for delivering therapeutics to a particular target.

### I. CROSS-REFERENCE TO RELATED APPLICATIONS

This application claims the benefit of priority to U.S. Provisional Application No. 63/184,769 filed May 5, 2021, which is hereby incorporated herein by reference in its entirety.

This invention was made with government support under Grant No. RP210206 awarded by the Cancer Prevention and Research Institute of Texas. The government has certain rights in the invention.

### II. BACKGROUND

Lung cancer, the deadliest form of cancer accounting for nearly 1.6 million cases around the world annually, has a less than 15% 5-year survival rate. Conventional treatment strategies such as surgery, radiation therapy (RT), and chemotherapy (CT) have limitations, including frequent relapse and severe side effects including hair loss, nausea, fatigue, and nerve damage. In addition, surgical removal of tumors is not always feasible in some lung cancer patients, for example, 30-40% of non-small-cell lung cancer (N-SCLC) patients consist of unresectable tumors. For several years, the main treatments have been RT and CT, and despite many technological advances, the five-year overall survival rate for these patients remains low. To reach the lung tumors while avoiding systemic organ toxicity, a more robust and cell specific drug delivery system needs to be envisaged. Novel drug delivery systems such as nanoparticle-based drug delivery can provide a drug to a tumor site and take on a cancer cell specific approach rather than a systemic one. This results in the delivery of the chemo drug to lung cancer cells for effective, localized chemotherapy to treat lung cancers.

Nanoparticle-based drug delivery applications have a vast potential for better effective treatment due to their unique properties compared with traditional drug formulations. Controlled and sustained release, increased stability and circulation time, enhanced bioavailability, altered organ distribution to reduce side effects, improved drug solubility, facilitated target site accumulation, and reduced administration frequency can be counted as advantages of nanoparticle-based drug delivery systems. Active targeted drug delivery utilizes targeting ligands via conjugating them on synthetic drug carrier nanoparticle surfaces to enable active seeking of targeted molecules on the intended cells. Moreover, active targeted nanoparticles today can be utilized not only as drug delivery agents but also as diagnostic tools via incorporation of specific dyes and/or imaging agents. In addition, they can be designed as hybrid systems called theragnostic nanoparticles to fulfill both of the above-mentioned duties at the same time.

Despite promising results, targeting ligand conjugated synthetic drug carriers have multiple challenges and limitations such as NP architecture, ligand target interactions, conjugation chemistry used, and host immune responses. Active and effective targeting mainly relies on carrier-target molecule interaction. Besides the above-mentioned limitations of active targeted drug carriers, another limitation is targeting efficiency of synthetic NP drug carrier systems. Structural characteristics of NPs have great effects on in vivo targeting. For instance, targeting ligands are very prone to shed off from surface during degradation in surface eroding polymeric carriers. Low density of ligands conjugated on the NP surface is another factor, resulting in an ineffective targeting. This is generally because of competitive inhibition, unwanted targeting ligand orientation and steric blockage of targeting moieties. On the contrary, increased uptake by the immune system, reducing adverse effects due to target binding kinetics, and minimizing non-specific binding to off-target cells are some of the results from having a high ligand density on the NP surface. What are needed are new compositions and methods for delivering nanoparticles and their therapeutic payload to the target site.

### III. SUMMARY

Disclosed are methods and compositions related to chimeric antigen receptor (CAR) engineered T lymphocyte membrane coated nanoparticles (CAR-T MNPs).

Also disclosed herein are chimeric antigen receptor (CAR) T cell membrane coated particle (CAR-T-MNP) comprising i) a T cell plasma membrane comprising a CAR on its cell membrane and ii) an engineered particle; wherein the engineered particle comprises a therapeutic agent (such as, for example, an anticancer agent, anti-inflammatory agent, or an antimicrobial (including, but not limited to an antiviral agent, an antibiotic, and/or an antifungal agent)).

In one aspect, disclosed herein are CAR-T-MNPs of any preceding aspect, wherein the CAR-T-MNP comprises a bi-specific CAR and/or two or more differently targeting CARs (a CAR comprising a first specificity and a second specificity and/or a first CAR comprising a first specificity and a second CAR comprising a second specificity). In one aspect, the first and second specificity can be to the same target.

Also disclosed herein are CAR-T-MNPs of any preceding aspect, wherein the CAR-T-MNP comprises a bi-specific CAR comprising a CAR-T plasma membrane with an intracellular facing binding moiety (such as, antibody, or fragment thereof such as a F(ab′)2, Fab′, Fab, Fv, or ScFv) and an extracellular facing binding moiety (such as, antibody, or fragment thereof such as a F(ab′)2, Fab′, Fab, Fv, or ScFv). In such aspects, the CAR comprises an extracellular binding moiety, a hinge region, a transmembrane domain (can be any transmembrane domain identified in the literature including, but not limited to CD8, CD4, CD28 transmembrane domains), an intracellular hinge region, and an intracellular binding moiety. In one aspect, the intracellular binding moiety and extracellular binding moiety are directed to the same target or different targets.

In one aspect, disclosed herein are CAR-T-MNP of any preceding aspect, wherein the engineered particle comprises poly(lactic co-glycolic) acid (PLGA).

Also disclosed herein are CAR-T-MNP of any preceding aspect, wherein the CAR comprises a first specificity specific for a cancer cell specific marker (such as, for example, carcinoembryonic antigen (CEA), EGFRVIII, IL-llRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin Bl, lectin-reactive AFP, Fos-related antigen 1, ADRB3, thyroglobulin, EphA2, RAGE-1, RUl, RU2, SSX2, AKAP-4, LCK, OY-TESl, PAX5, SART3, CLL-1, fucosyl GM1, GloboH, MN-CA IX, EPCAM, EVT6-AML, TGS5, human telomerase reverse transcriptase, plysialic acid, PLAC1, RUl, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, HER2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and/or mesothelin; or the CAR is specific to peptides processed from target list that are presented by Human Leukocyte Antigens (pHLAs).

In one aspect, disclosed herein are CAR-T-MNPs of any preceding aspect, wherein the therapeutic agent comprises a small molecule, RNAi, gene vector, peptide, polypeptide, protein, or antibody.

Also disclosed herein are methods of targeting the delivery of a therapeutic agent to a target site in a subject in need thereof comprising administering to the subject any of the CAR-T-MNP of any preceding aspect.

In one aspect disclosed herein are methods of treating, inhibiting, reducing, ameliorating, and/or preventing a cancer and/or metastasis (such as, for example, non-small cell lung cancer or breast cancer), an inflammatory condition, and/or a microbial infection (such as, for example a viral, bacterial, fungal, or parasitic infection) in a subject comprising administering to the subject any of any preceding aspect.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the overall view of proposed CAR-T-MNPs for targeted N-SCLC therapy.

FIG. 2 shows flow cytometry confirmation of anti HER-2 antibodies in hybridoma cell culture supernatants.

FIGS. 3A, 3B and 3C show plasmid purification. FIG. 3A shows Line 1: pLP/VSVG, Line 2: 4D5 Anti-HER-2 CAR. FIG. 3B shows Line 1: psPAX, Line 2: EcoRI digested. FIG. 3C shows C) Line 1: mCherry fluorescent coding plasmid, Line 2: EcoRI digested plasmids L: 1 kb step DNA ladder.

FIG. 4 shows Lentiviral vectors produced in Lenti-X 293T packaging cell line.

FIG. 5 shows Viral titer measurement via p24 ELISA lentiviral tittering study.

FIGS. 6A and 6B show collected viral particle transduction on HEK293 T cells, concentration applied to cells with +8 μg/ml Polybrene for 48 hours. 6A: DIC and 6B: Red channel).

FIGS. 7A and 7B show confirmation of anti-HER2 ScFv transduction on Jurkat T-cells by 7A) Fluorescent microscopy imaging of mCherry expression on the transduced and sorted cells brightfield channel (left) fluorescent channel (right). FIG. 7B shows flow cytometric confirmation of Jurkat cell transduction, anti-HER2 CAR transduced cells (red) showing a distinctive shift against both mcherry and c-myc compared to the non-transduced Jurkat cell (black).

FIGS. 8A, 8B, and 8C show physiological Characterization of CAR-T-MNPs. FIG. 8A shows drug release comparison of CAR-T-MNP vs bare PLGA NPs. FIG. 8B shows blood clotting trend of CART-MNPs compared to saline group. FIG. 8C shows hemolysis graph of CAR-T-MNPs.

FIGS. 9A, 9B, 9C, 9D, and 9E show the physiochemical characterization of CAR-T-MNPs. FIG. 9A shows TEM image of CAR-T-MNPs. FIG. 9B shows size, zeta potential and polydispersity chart of NP formulations. FIG. 9C shows drug release kinetics of CAR-T-MNPs in PBS buffer. Anti-HER2 presence and functionality confirmation of 9D) CAR-T-MNPs by flow cytometry against myc protein (CAR-T-MNPs (red), uncoated PLGA NPs (Black)). FIG. 9E shows CAR-T cell by Western blot shows distinctive band correlating to anti-HER2 CAR ScFv for CAR-T cells. No such band was observed for Jurkat-T cells.

FIGS. 10A and 10B show the confirmation of CAR-T membrane coating (stained with DiD dye) on PLGA Nanoparticle (Coumarin-6) by 10A Flowcytometry, PLGA NPs (FITC channel) Stained exterior membranes (Red channel) 10B Fluorescent microscopy merged image (TRITC-channel).

FIGS. 11A and 11B show Flow cytometric coating efficacy statistics of CAR-T-MNPs.

FIGS. 12A, 12B, and 12C show coumarin-6 PLGA NPs extruded with CAR-T-MNPs and stained with lipophilic DiD dye. 12A) PLGA NPs (FITC channel) 12B) Stained exterior membranes (Red channel) 12C) merged image (TRITC-channel).

FIGS. 13A and 13B show anti-HER-2 existence confirmation on isolated and extruded CAR-T-MNPs.

FIGS. 14A, 14B, and 14C show the hemocompatibility of CAR-T-MNPs in vitro. FIG. 14A shows blood clotting kinetics/effects when blood incubated with different concentrations (250 μg/ml-1000 μg/ml) of CAR-T-MNPs. FIG. 14B shows hemolysis level when blood incubated with CAR-T-MNPs with ranged concentrations (0 μg/ml-1000 μg/ml). FIG. 14C shows cyto-compatibility analysis of CAR-T-MNPs on AT1 cells to determine their in vitro cytotoxic effect on different concentrations (0 μg/ml-1000 μg/ml). The data are shown as mean (SD (n=4))

FIG. 15A shows spectrophotometric analysis of different NP group's uptake trend in HER2 expressing A549 cells. The data are shown as mean (SD (n=4), ***P<0.001, ****P<0.0001).

FIG. 15B shows fluorescent uptake images of PLGA NPs, Jurkat-T-MNPs and CAR-T-MNPs by A549.

FIGS. 16A and 16B show in vitro therapeutic efficiency (cell killing) properties of CAR-T-MNPs. 16A) SKOV-3 16B) A549 cells exposed to free cisplatin, cisplatin-loaded PLGA NPs, Jurkat-T-MNPs & CAR-T-MNPs (both loaded with cisplatin) for 48 hours. Cell viability was quantified via MTT assays after exposure (SD (n=4), ****P<0.0001).

FIG. 17 shows in vitro tumor cell killing properties of CAR-T-MNPs. MDA-MB-231 (HER-2 negative); A549 (HER-2 positive) and SKOV-3 cells (HER-2 positive) exposed to free afatinib, PLGA NPs, Jurkat-T-MNPs, anti HER-2 PLGA NPs and CAR-T-MNPs (all NPs loaded with afatinib) for 48 hours. Cell viability was quantified via MTT assays after the exposure.

FIG. 18 shows fluorescent live/dead assay images of all treatment groups.

FIG. 19 shows Live/dead assay image analysis quantification of all treatment groups.

FIGS. 20A, 20B, and 20C show in vivo biodistribution (targeting efficiency) studies. FIG. 20A shows in vivo measurement of average color intensity of tumors/mm2 area on animal groups (CAR-T-MNPs, Jurkat-T-MNPs, PLGA NPs, and saline). FIG. 20B shows representative organ images of all biodistribution study groups. FIG. 20C shows accumulation efficiency of fabricated nanoparticle systems in individual organs and tumors. (SD (n=4), ****P<0.0001)

FIGS. 21A, 21B, 21C, and 21D show in vivo therapeutic efficacy of CAR-T-MNPs on subcutaneous A549 tumor implanted in nude mice compared to Jurkat-T-MNPs and free cisplatin. A) Tumor volume delay analysis for 2 weeks after treatment of mice with different groups. B) Weight change of the mice during the duration of treatment. C) Animal survival analysis of tumor implanted mice (sacrificed when tumor growth >20 mm in length/width). D) Histology analysis (H & E staining) of tumor tissue extracted from mice after experiment endpoint. The data are shown as mean (SD (n=3))

FIG. 22 shows a schematic diagram of bispecific double sided chimeric transmembrane receptor construction. (Left) Plasmid shown with cloning site between BstEII and NotI where anti-HER2 double side chimeric antigen related genes will be inserted. (Right) Anti HER2 double sided chimeric transmembrane receptor coding lentiviral plasmid. Anti her-2 ScFv's (either VL-VH or VH-VL formation) Hinge domain (either CD3, CD28 or CD8), transmembrane domain (either CD3, CD28 or CD8), linkers (GSTSGSGKPGSGEGS or (G₄S)₃), BstEII and NotI, restriction sites.

FIG. 23 shows confirmation of Double-sided CAR-T cell by Western blot shows distinctive band (75 KDa) correlating to double-sided anti-HER2 CAR ScFv. No such band was observed for Jurkat-T cell.

FIG. 24 shows the isolation, expansion and lentiviral transduction of BiCAR cells.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular chimeric antigen receptor (CAR) engineered T lymphocyte membrane coated nanoparticles (CAR-T MNP) is disclosed and discussed and a number of modifications that can be made to a number of molecules including the CAR-T MNP are discussed, specifically contemplated is each and every combination and permutation of CAR-T MNP and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Particle based drug delivery has not yet fully been controlled and applied clinically for reasons of the opsonization, non-specific clearance, relatively short circulation time, and limitations of fabricating actively targeted drug carriers. Blood component interactions of these synthetic NPs, on the other hand, is another factor that affects the NP's fate in the body. This interaction creates a layer on the surface of NPs called “protein corona” (PC). The PC is responsible for cellular uptake, physiological features, immune responses, and targeting efficiency of the particles. For these reasons, using very well characterized carrier materials and identifying the blood component interactions as well as their PC composition are needed for the targeted drug delivery applications. Above-mentioned short-comings in the synthetic particle-based drug delivery field make the use of biomimetic materials to create advanced drug delivery systems more and more popular.

Bacteria or virus-based carriers, hybrid nano-bio systems, and cell based drug delivery vehicles have been reported to have applications for biomimetic targeted drug delivery. Among these, development of cell membrane coated synthetic polymeric nanoparticles as drug carriers also has broad application in the field. Furthermore, this type of cell based biomimetic drug delivery application might provide a new way for individualized medicine applications by applying the patients' own cells for their own treatments.

Current applications of lymphocyte membrane coated carriers include neutrophil membrane coated nanoparticles for targeting metastatic tumors cells, shows the expression of ligands for the adhesive proteins that are expressed on neutrophil membranes. In another application, cytotoxic T-lymphocyte coated NPs were used for targeting and treating gastric cancer. Our group has shown that a T-cell hybridoma membrane expressing a melanoma-specific anti-gp100/HLA-A2 T-cell receptor when coated onto trametinib loaded PLGA, showed more than a two-fold increase in tumor retention compared to just nanoparticles. In addition, T-lymphocyte membrane-encapsulated PLGA NPs were able to avoid being segregated by lysosomes and retained their lymphocyte coating on the NPs while trapped in the endolysosomal compartments. Furthermore, tumor-associated carcinoembryonic antigen (CEA) chimeric antigen receptor (CAR) engineered jurkat cells were actively accumulated in vivo on liver tumors compared to normal jurkat cells in cell-based viral therapeutic delivery. Therefore, the strategy of targeting molecule (CAR, TCR, and ScFv) engineered jurkat cell membrane coated drug delivery is a promising approach for the biomimetic drug delivery field, going forward. Furthermore, this type of cell based biomimetic drug delivery application provide a new way for individualized medicine applications by applying the patients' own cells for their treatments.

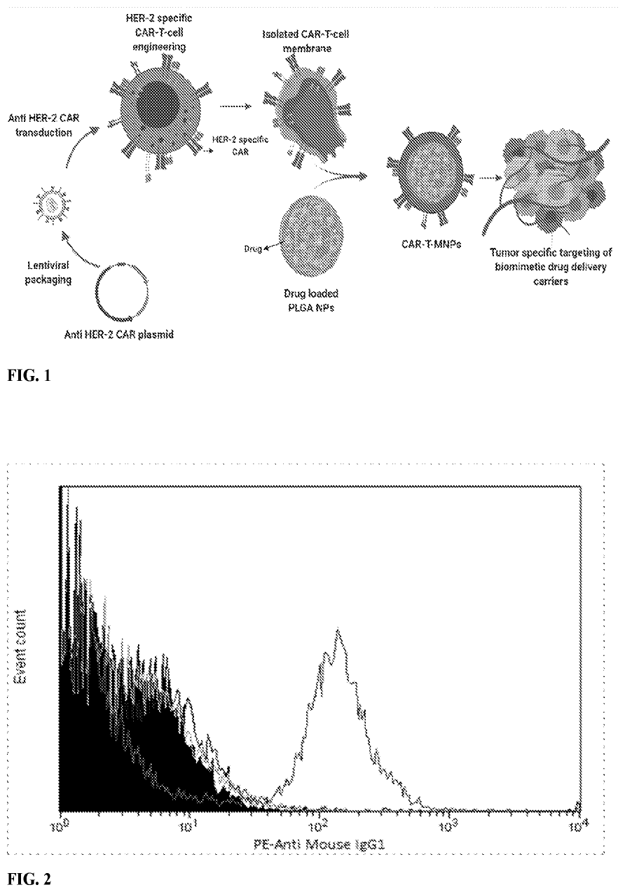

Here we describe a unique approach, combining chemo-drug loaded PLGA NPs with CAR-T cell membranes extracted from genetically engineered human Jurkat lymphocytes to target HER-2 positive lung cancer for therapy. The overall design is presented in FIG. 1. Co-extrusion methods were used to fabricate targeting nanoparticles with prolonged circulation and avoiding systemic clearance and undesired PC formation. Thus, there was an increased ability of anti-HER-2 CAR-T-MNPs to migrate and localize at tumor sites that is not otherwise achievable through current modification and bioconjugation techniques of targeting moieties on NPs. PLGA NPs are chosen due to their sustained drug release profile, biocompatibility and biodegradability, proven efficacy, both FDA and European Medical Agency approval to be used as chemo drug carrier systems and wide variable drug loading capacities. Cisplatin is an FDA approved chemotherapeutic drug used in the study, which is effective against lung and breast cancer. These new nano drug carriers, called CAR-T-MNPs, are produced by camouflaging PLGA NPs with HER-2 ScFv expressing cellular membranes isolated from genetically modified lymphocytes. Several in vitro and in vivo experiments were performed to determine if CAR-T-MNPs could increase circulation time and actively accumulate on HER-2 expressing tumor tissues.

In one aspect, disclosed herein are chimeric antigen receptor (CAR) T cell membrane coated particles (CAR-T-MNP) comprising i) a T cell plasma membrane comprising a CAR on its cell membrane and ii) an engineered particle; wherein the engineered particle comprises a therapeutic agent (such as, for example, an anticancer agent, anti-inflammatory agent, or an antimicrobial (including, but not limited to an antiviral agent, an antibiotic, and/or an antifungal agent)). It is understood and herein contemplated that the primary use of the disclosed CAR-T-MNPs is to deliver a therapeutic agent. Thus, in one aspect, disclosed herein are methods of targeting the delivery of a therapeutic agent to a target site in a subject in need thereof comprising administering to the subject any of the CAR-T-MNP disclosed herein.

It is understood and herein contemplated that to direct the activity of the therapeutic agent, the CAR can have specificity for a particular cell type, tissue type, or cell surface molecule expressed by a cancer cell or cell with a mutation. For example, to target cancer cells, in one aspect, disclosed herein are CAR-T-MNPs, wherein the CAR comprises a first specificity specific for a cancer cell specific marker (such as, for example, carcinoembryonic antigen (CEA), EGFRVIII, IL-llRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin Bl, lectin-reactive AFP, Fos-related antigen 1, ADRB3, thyroglobulin, EphA2, RAGE-1, RUl, RU2, SSX2, AKAP-4, LCK, OY-TESl, PAX5, SART3, CLL-1, fucosyl GM1, GloboH, MN-CA IX, EPCAM, EVT6-AML, TGS5, human telomerase reverse transcriptase, plysialic acid, PLAC1, RUl, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and/or mesothelin; or the CAR is specific to peptides processed from target list that are presented by Human Leukocyte Antigens (pHLAs). Similarly, the CAR can express a receptor that is specific for a cell surface molecule unique to any cell in the body. A non-limiting less of cells that can be targeted by the disclosed CAR-T-MNPs include, but are not limited to, B cells, epithelial cells, neurons, smooth muscle cells, cardiac muscle cells, hepatocytes, langerhans cells, granculocytes, chondrocytes, osteoblasts, osteoclasts, islet cells (alpha or beta), corticotropes, gonadoctropes, lactotropes, melanotropes, somatotropes, keratinocytes, melanoctyes, and adipocytes.

In some aspect, the CAR-T-MNP can comprise a bi-specific CAR or two or more differently targeting CARs (a CAR of a first specificity and a CAR of a second specificity). In one aspect, the first and second specificity can be to the same target. For example, the CAR-T-MNP can comprise a CAR-T plasma membrane with an intracellular facing binding moiety (such as, antibody, or fragment thereof such as a F(ab′)2, Fab′, Fab, Fv, or ScFv) and an extracellular facing binding moiety (such as, antibody, or fragment thereof such as a F(ab′)2, Fab′, Fab, Fv, or ScFv). In such aspect, the CAR comprises an extracellular binding moiety, a hinge region, a transmembrane domain, an intracellular hinge region, and an intracellular binding moiety. In one aspect, the intracellular binding moiety and extracellular binding moiety are directed to the same target.

The disclosed CAR-T-MNPs comprise an engineered particle which comprises a therapeutic agent. As disclosed herein, the engineered particle can be produced using a biocompatible polymer such as, for example, poly(lactic co-glycolic) acid (PLGA). In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

It is understood and herein contemplated that in addition to the biodegradable polyesters and polyanhydrides disclosed above, the biocompatible polymers used to construct the disclosed engineered particles (such as, for example a nanoparticle or microparticle) used in the disclosed CAR-T-MNP can be any biocompatible polymer known by those of skill in the art. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

The block copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one aspect, the core polymer can be flanked by polypeptide blocks.

As noted throughout this disclosure, the CAR-T-MNPs disclosed herein comprise an engineered particle which is designed to deliver a therapeutic agent (such as, for example, an antibiotic, an antiviral agent, an antifungal agent, an anti-inflammatory agent, and/or an anti-cancer agent) to a target cell or tissue type. In one aspect, disclosed herein are CAR-T-MNPs, wherein the therapeutic agent comprises a small molecule, RNAi, gene vector, peptide, polypeptide, protein, or antibody.

In one aspect, the therapeutic agent can comprise an anti-cancer agent. The therapeutic agent can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate), a PD-1 inhibitor, a PD-L1 inhibitor, or CTLA-4 inhibitor (such as, for example, nivolumab, pembrolizumab, pidilizumab, BMS-936559, Atezolizumab, Durvalumab, or Avelumab), or any salts, esters, amides, prodrugs, proagents, conjugates, active metabolites, isomers, fragments, and/or analogs thereof.

Similarly, the engineered particle can comprise an antiviral agent. A non-limiting list of antiviral agents for use in the disclosed CAR-T-MNPs include, but are not limited to, acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; levetiracetam; tramadol; or any of their isomers tautomers, analogs, polymorphs, solvates, derivatives, or pharmaceutically acceptable salts.

In one aspect, the engineered particle can comprise an antibiotic. A non-limiting list of antibiotics for use in the disclosed CAR-T-MNPs include, but are not limited to, amikacin, gentamicin, kanamycin, neomycin, tobramycin, paramomycin, streptomycin, spectinomycin, geldanamycin, herbimycin, rifaximin, loracarbef, ertapenem, doripenem, imipenem, meropenem, cefadroxil, cefazolin, cephradine, cephapirin, cephalothin, cefalexin, cefprozil, loracarbef, cefonicid, cemetazole, cefamandole, cefdinir, cefoperazone, cefotaxime, cefpodoxime, ceftazidime, ceftabiprole, Ceftaroline fosamil, vancomycin, dalbavancin, lincomycin, cleocin, oritavancin, dalbavancin, daptomycin, spriamycin, linezolid, penicillin, amoxicillin, ampicillin, dicloxacillin, methicillin, oxacillin, bacitracin, colistin, polymyxin B, ciprofloxacin, enoxacin, Gemifloxacin, levofloxacin, ofloxacin, mefanide, sulfacetamide, sulfadiazine, sulfamethizole, doxycycline, tetracycline, oxytertracycline, cycloserine, pyrazinamide, rifampicin, isoniazid, dapsone, clofazimine, pyrszinamide, or any of their isomers tautomers, analogs, polymorphs, solvates, derivatives, or pharmaceutically acceptable salts.

In one aspect, the engineered particle can comprise an antifungal agent. A non-limiting list of antibiotics for use in the disclosed CAR-T-MNPs include, but are not limited to, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, propiconazole, ravuconazole, terconazole, or any of their isomers tautomers, analogs, polymorphs, solvates, derivatives, or pharmaceutically acceptable salts.

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHOD OF TREATING CANCER

The disclosed compositions can be used to treat any disease where uncontrolled cellular proliferation occurs such as cancers. Thus, in one aspect, disclosed herein are methods of treating, inhibiting, reducing, ameliorating, and/or preventing a cancer and/or metastasis (such as, for example, non-small cell lung cancer or breast cancer), an inflammatory condition, or a microbial infection (such as, for example a viral, bacterial, fungal, or parasitic infection) in a subject comprising administering to the subject any of any preceding aspect.

A non-limiting list of different types of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

Therapeutic agents that can be encapsulated by the disclosed CAR-T-MNPs for treatment of a cancer in any of the methods disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate), a PD-1 inhibitor, a PD-L1 inhibitor, or CTLA-4 inhibitor (such as, for example, nivolumab, pembrolizumab, pidilizumab, BMS-936559, Atezolizumab, Durvalumab, or Avelumab), or any salts, esters, amides, prodrugs, proagents, conjugates, active metabolites, isomers, fragments, and/or analogs thereof.

### D. METHODS OF TREATING MICROBIAL INFECTIONS

It is understood and herein contemplated that the disclosed CAR-T-MNPs can deliver any therapeutic agent and are not limited to a particular, indication. For example, the disclosed CAR-T-MNPs can be used to deliver an antimicrobial to treat a microbial infections. In one aspect disclosed herein are methods of treating, inhibiting, reducing, ameliorating, and/or preventing a microbial infection (such as, for example a viral, bacterial, fungal, or parasitic infection) in a subject comprising administering to any of the CAR-T-MNPs disclosed herein.

For example, disclosed herein are methods of treating, inhibiting, reducing, ameliorating, and/or preventing a microbial infection, wherein the microbial infection is a viral infection, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, 20) Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

Where the treatment methods is designed to treat a viral infection, it is understood an herein contemplated that the therapeutic agent comprises an antiviral agent selected from the group comprising acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; levetiracetam; tramadol; and/or any of their isomers tautomers, analogs, polymorphs, solvates, derivatives, or pharmaceutically acceptable salts.

Also disclosed herein are methods of treating, inhibiting, reducing, ameliorating, and/or preventing a microbial infection, wherein the microbial infection is a bacterial infection, wherein the bacterial infection comprises an infection of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species. Where the treatment methods is designed to treat a bacterial infection, it is understood an herein contemplated that the therapeutic agent comprises an antibiotic selected from the group comprising amikacin, gentamicin, kanamycin, neomycin, tobramycin, paramomycin, streptomycin, spectinomycin, geldanamycin, herbimycin, rifaximin, loracarbef, ertapenem, doripenem, imipenem, meropenem, cefadroxil, cefazolin, cephradine, cephapirin, cephalothin, cefalexin, cefprozil, loracarbef, cefonicid, cemetazole, cefamandole, cefdinir, cefoperazone, cefotaxime, cefpodoxime, ceftazidime, ceftabiprole, Ceftaroline fosamil, vancomycin, dalbavancin, lincomycin, cleocin, oritavancin, dalbavancin, daptomycin, spriamycin, linezolid, penicillin, amoxicillin, ampicillin, dicloxacillin, methicillin, oxacillin, bacitracin, colistin, polymyxin B, ciprofloxacin, enoxacin, Gemifloxacin, levofloxacin, ofloxacin, mefanide, sulfacetamide, sulfadiazine, sulfamethizole, doxycycline, tetracycline, oxytertracycline, cycloserine, pyrazinamide, rifampicin, isoniazid, dapsone, clofazimine, pyrszinamide, and/or any of their isomers tautomers, analogs, polymorphs, solvates, derivatives, or pharmaceutically acceptable salts.

In one aspect, disclosed herein are methods of treating, inhibiting, reducing, ameliorating, and/or preventing a microbial infection, wherein the microbial infection is a fungal infection, wherein the fungal infection comprises an infection of Candida albicans, Cryptococcus neoformans, Histoplama capsulatum, Aspergillus fumigatus, Coccidiodes immitis, Paracoccidioides brasiliensis, Blastomyces dermitidis, Pneumocystis carinii, Penicillium marneffi, or Alternaria alternata. When the microbial infection is a fungal infection, the therapeutic agent can comprises an antifungal agent selected from the group consisting of bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, propiconazole, ravuconazole, terconazole, and/or any of their isomers tautomers, analogs, polymorphs, solvates, derivatives, or pharmaceutically acceptable salts.

Also disclosed herein are methods of treating a microbial infection wherein the microbe is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### E. METHODS OF TREATING AN INFLAMMATORY CONDITION

The disclosed CAR-T-MNPs can also be used for the targeted deliver of anti-inflammatory agents to treat inflammatory conditions and/or inflammatory symptoms associated with an injury or microbial infection. Thus, in one aspect, disclosed herein are method of treating, inhibiting, reducing, ameliorating, and/or preventing an inflammatory condition (including, but not limited to inflammation resulting from myocardial infarction, abrasion, puncture, laceration, contusion, blunt force trauma, ischemia, hemorrhagic stroke, surgery, transplant, sunburn, chemical burn, high temperature burn, low temperature burn, or injury due to microbial infection) in a subject comprising administering to the subject any of the CAR-T-MNP disclosed herein.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Herceptin-Based CAR Transduced T Lymphocyte Membrane Camouflaged Nanoparticles (CAR-T MNP). A New Approach to Enhance Drug Targeting and Circulation Time in Lung Cancer

### a) Results

### (1) Fabrication, Purification and Characterization of 4D5 Anti HER-2 Antibodies

4D5 hybridoma cell line was tested for the secreted antibody existence in culture supernatant. For this, 1×10⁶ A549 cells were stained for flow cytometric analysis. As seen in the flow cytometry histogram (FIG. 2), 4D5 cell culture supernatant treated cells with anti-HER2 staining show a significant shift. Therefore, the existence of anti HER-2 antibody in the supernatant was demonstrated, and thereby antibody collection and purification steps were performed. The purified antibody product was also validated.

After purification steps, positive fractions were selected based on their 280 nm absorbance on nanodrop and total concentration of purified antibody was calculated. Following, obtained antibodies were tested to assess their binding functionality against A549 cells. For this binding study, A549 cells were stained with purified antibodies and PE-anti mouse secondary antibody. Purified antibodies showed a significant shift in PE channel for the stained A549 group.

### (2) Plasmid Purification, Digestion and Confirmation

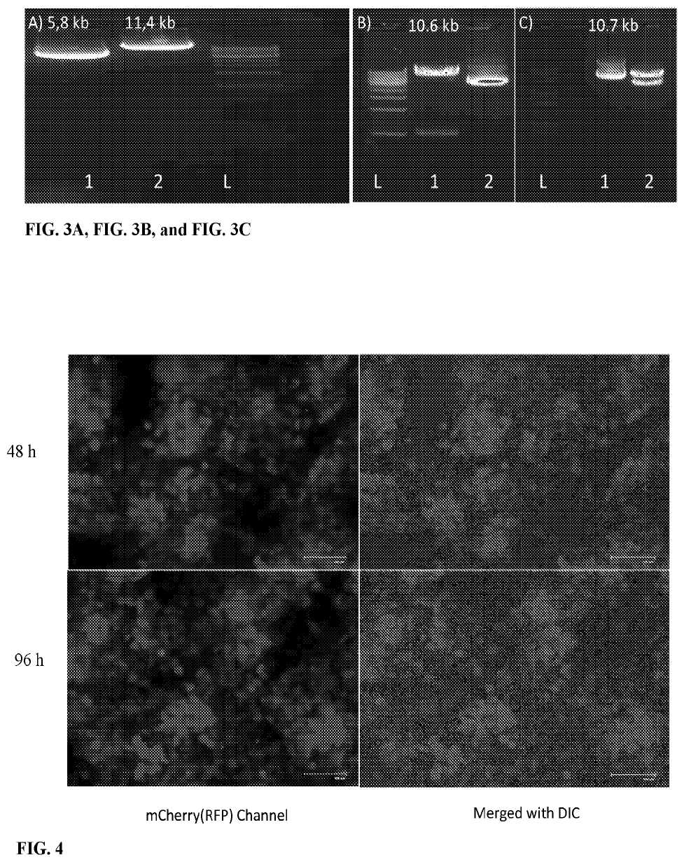

Transformed bacterial cultures were grown in lab, and PCR confirmed positive colonies were expanded further and used for purifying plasmids (Qiagen Mega plasmid DNA isolation kit). These purified plasmids were confirmed by PCR, enzyme digestion (EcoRI) and gel electrophoresis (by size) as shown in FIG. 3.

### (3) Lentiviral Vector Production

Lipid-based plasmid DNA transfection methods were followed for lentiviral transfection studies. Lenti-X 293T cells grown in 6 well plates were exposed to psPAX+pLP/VSVG+ with 4D5 anti-HER-2 CAR and/or mCherry fluorescent protein coding plasmids together as equimolar mass in total of 3 μg. After transfection, constitutive mCherry fluorescence (FIG. 4) was observed, and culture supernatant was collected over the course of 3 days. Cell culture supernatants were then used to transduce Jurkat T-cells to express mCherry and anti HER-2 CAR proteins.

### (4) Generation of CAR-T Cell

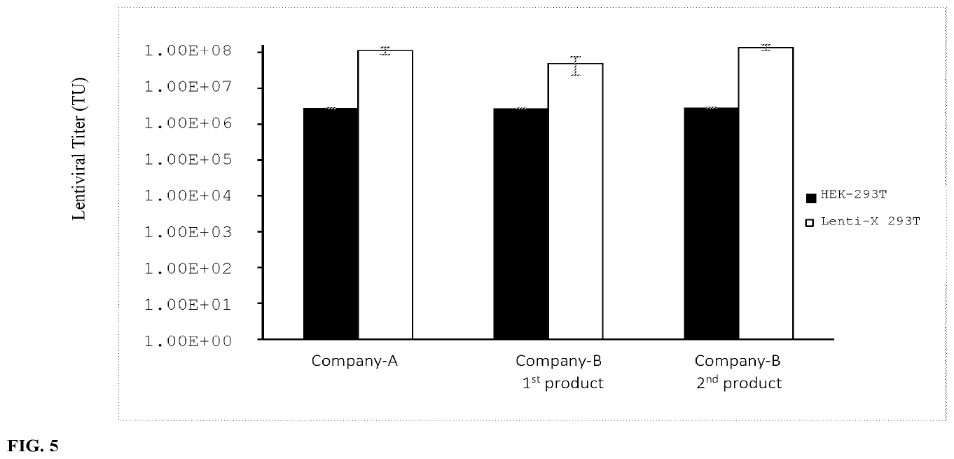

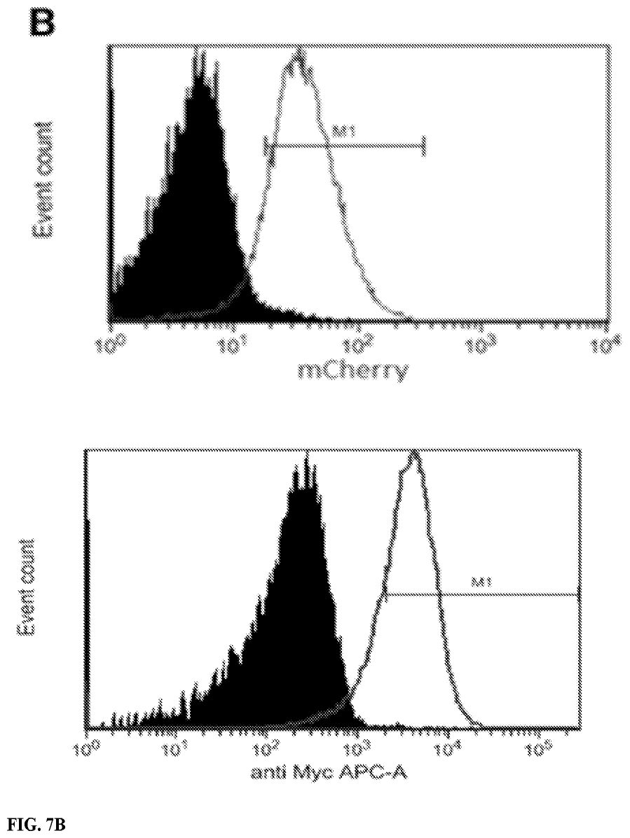

Plasmids including packaging psPAX (addgene plasmid #12260)+pLP/VSVG (Invitrogen) and expression plasmids 4GALUAS_tBFP_mCherry (addgene plasmid #79130)+anti HER2 SynNotch (addgene plasmid #85424) were used for generating CAR (or anti-HER2) targeting HER2 cancer cells. The Lenti-X 293T lentiviral packaging cell line was used to generate lentiviral particles, which were employed in our experiments for transduction purposes. Plasmids were transfected into Lenti-X 293T cells via lipofectamine with a high transfection efficiency (˜100%) evaluated using red fluorescence of mcherry reporter proteins. We then optimized the lentiviral particles produced by transfected Lenti-X 293T cells using viral titer kits (FIG. 5), and multiplicity of infection (MOI) of 1:10 (T cell: viral particles) was calculated to match the viral titer requirement for jurkat cell line which undergoes transduction. Transduction was observed through internal mcherry expression, which showed a distinctive shift in transduced jurkat cells (FIG. 7A).

As shown in FIG. 7B, jurkat cells were successfully transduced to express anti-HER2 CAR receptor on their cell surface. We included a c-myc tag towards the end of the extracellular CAR construct for sorting and CAR characterization. After staining for myc tag, a distinctive shift was seen in the anti myc-APC channel of the transduced T-cell group compared to the non-transduced anti-myc tag stained group. In total, approximately 40-50% of the transduced cell population was found to express anti-HER2 CAR receptor on the surface.

Transduced cells were sorted against c-myc antibody using a BD melody cell sorter for positive clones of CAR-T cells with a positive clone of 70-80% from the whole population. Expression stability of sorted CAR-T jurkat cells was assessed for two weeks. The stability study revealed that 93-97% of CAR-T cells still maintain the expression of engineered CAR. After this confirmation, cells were further cultured for their membrane isolation as needed.

### (5) Lentiviral Transduction of Jurkat T-Cells

Collected viral particles were applied to cells with +8 μg/ml Polybrene for 48 hours. After viral transduction, fluorescent microscopic pictures were taken of anti HER-2+mCherry expressing Jurkat cells. After transduction confirmation via fluorescent microscopy, transduction of cells was confirmed and characterized via flow cytometry. In this case, anti-CD3-PE antibody (PE channel) was used to characterize Jurkat cells, whereas internal mCherry fluorescent protein (PerCP-Cy5.5 channel) was used for transfection.

### (6) Anti HER-2 TCR Receptor Confirmation on Jurkat Cell Surface

After confirmation of reporter plasmid transduction and stable mCherry protein expression, the same set of experiments was used for anti her-2-CAR receptor encoding transfer plasmid transduction studies. In the extracellular portion of anti HER-2 CAR receptor, a small tag called c-myc was placed as an extra sequence in the plasmid to detect the receptor easily on the cell surface. In this case, instead of fluorescent protein mCherry, Alexa fluor 647 anti myc-tag antibody was used. As shown in FIG. 7, Jurkat cells were successfully transduced to express anti HER-2 CAR receptor on their cell surface. They were also characterized by their CD3 expression as well. Again, a distinctive shift was seen in the anti myc-APC channel compared to the non-transduced anti-myc tag stained group. As transduction efficacy, around 40-50% of all the cell population were found to express anti HER-2 CAR receptor on the surface.

(7) Transduced T-Cell Sorting (mCherry Based)

Transduced cells were sorted via BD melody cell sorter. For the sorting experiment, mCherry was used to sort out mCherry transduced cells from the negative population. Almost half a million Jurkat cells were sorted by using PERCP-Cy5.5 channel with around 70-80% efficiency. Cells had been sorted with BD melody using aseptic techniques and negative/positive fractions were cultured further. Later, fluorescent images were taken of cultured positive and negative cell fractions to show sorting efficacy of transduced T-cells.

As shown in FIG. 7, sorted positive fraction shows mCherry expression compared to the negative sorted group. The next step was to check whether transduced Jurkat cells were these plasmid ORFs stable in a long-term. For this, transduced, sorted cells were cultured at least two weeks to confirm their expression stability. Their stable gene expressions were confirmed via fluorescent microscopy and flow cytometry. After this confirmation, cells were further sorted and characterized by their HER-2 expressions and further cultured for their membrane isolation.

(8) Long Term Stable mCherry ORF Expression after Sorting

Cells after two weeks of subculture were imaged under the fluorescent microscopy, and their mCherry expression was also confirmed via flow cytometry.

As shown in FIG. 7, mCherry transduced Jurkat cells were able to express transduced genes over two weeks. In fluorescent microscopy, almost all the cells were showing mCherry expression (red). In flow cytometry result, cells again showed a distinctive shift compared to the non-transduced Jurkat cell group and having more than 87% mCherry positive cell population.

### (9) Transduced T-Cell Sorting (HER-2 Expression Based)

In the second set of cells sorting experiments, anti HER-2 expressing cells were sorted by using Alexa fluor 647 anti-myc tag antibodies. More than half a million Jurkat cells were again sorted by using APC channel with around 93-97% efficiency.

(10) Long Term Stable mCherry & Anti-HER-2 CAR Expression after Sorting

Stable anti HER-2 CAR expression in the cultured cells after 2 weeks of expansion was confirmed via flow cytometry. As seen in FIG. 7, mCherry and anti HER-2 CAR transduced Jurkat cells were able to express transduced genes that were stable after two weeks.

### (11) Physical Characterization of CAR-T-MNPs

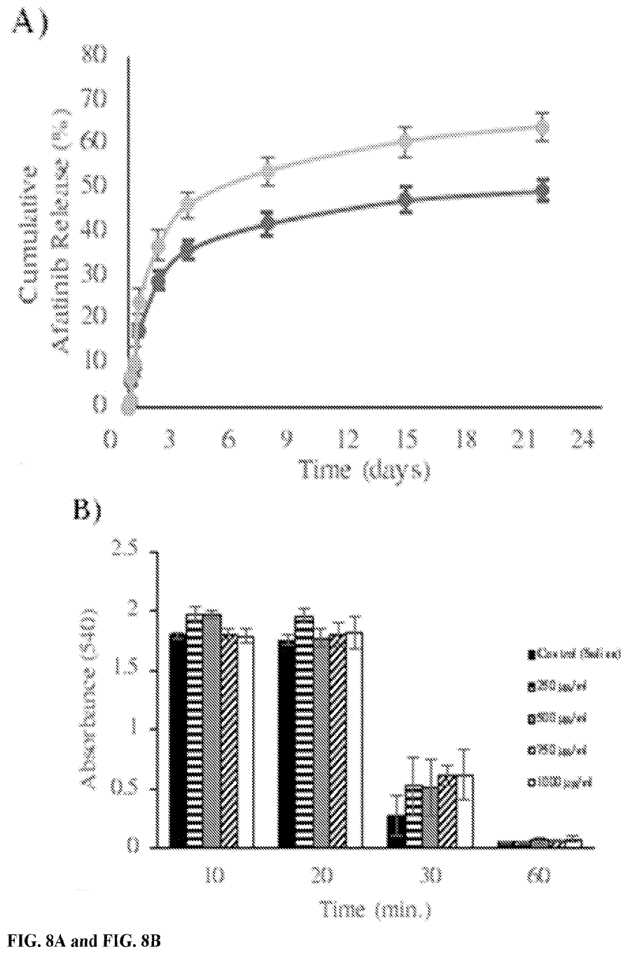

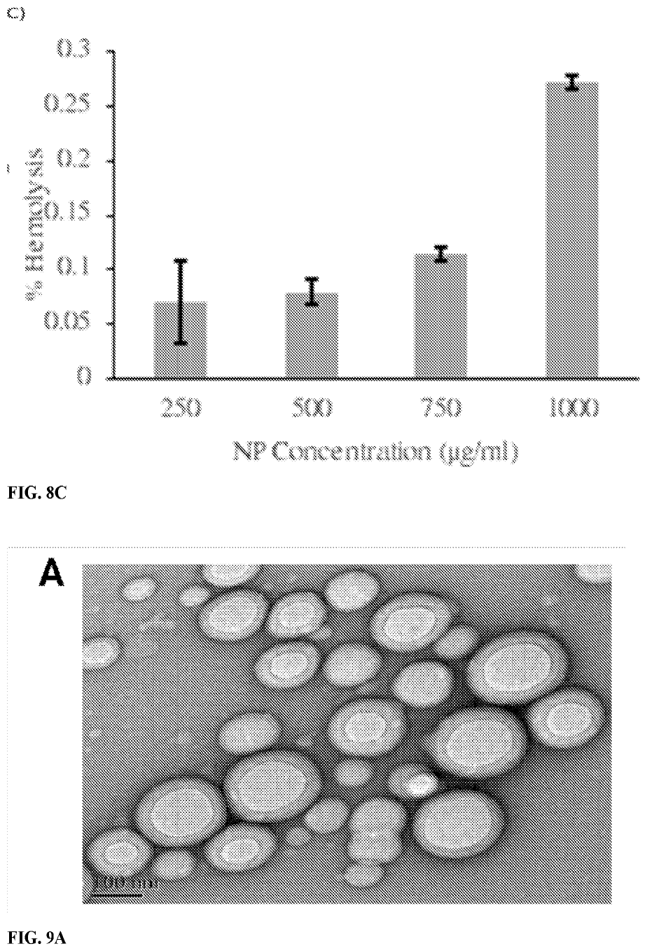

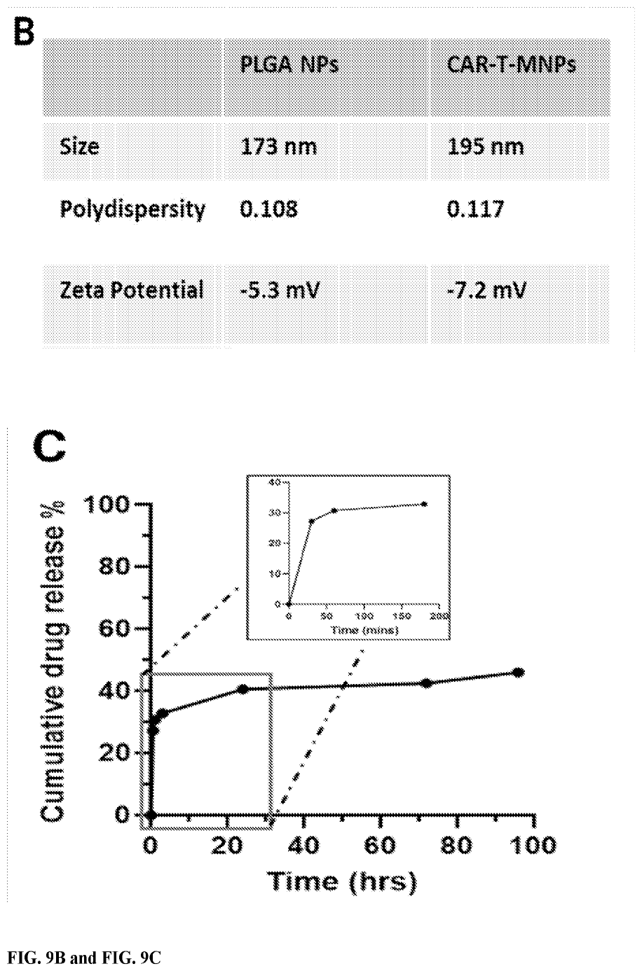

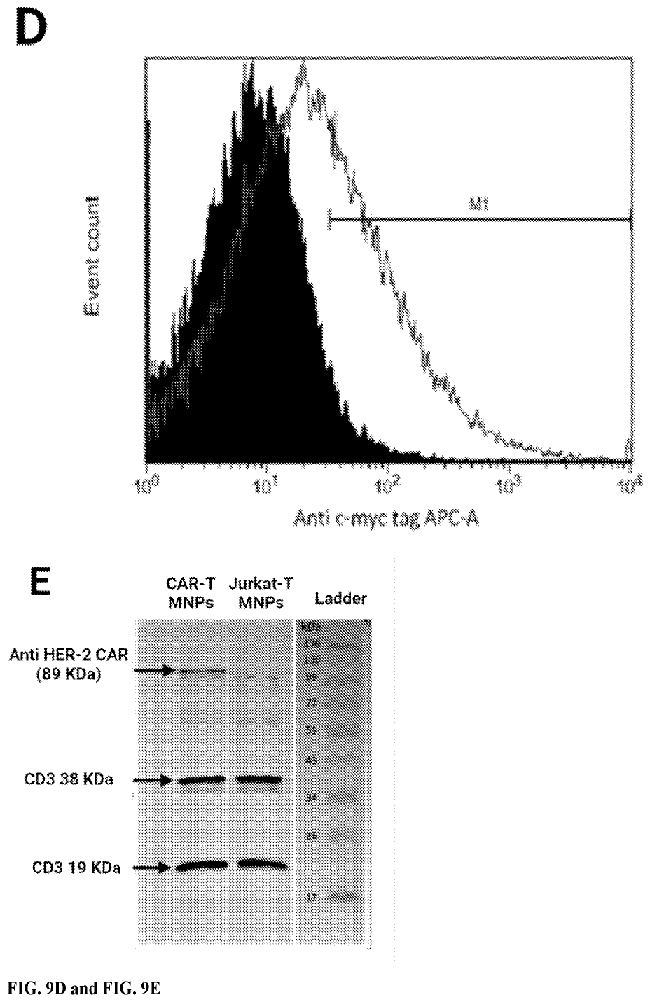

CAR-T-MNPs were synthesized with a two-step process: 1-CAR-T membrane isolation from genetically engineered T-cells and co-extrusion with afatinib loaded PLGA NPs. First, anti HER-2 CAR-T-cell membranes were collected via hypotonic membrane extraction method. Second, afatinib loaded PLGA nanoparticles were synthesized via double emulsion method. Isolated anti HER-2 CAR-T-MNPs were then reconstituted and co-extruded through a 400-nm polycarbonate membrane with afatinib loaded PLGA NPs to form CAR-T-MNPs. The extruded NPs were hemocompatible, and the blood clotting graph and in vitro drug release profile are also shown in FIG. 8. TEM images of both bare PLGA NPs and CAR-T-MNPs revealed core shell or onion like structure on the CAR-T-MNP group while bare PLGA NPs were shown as spheres (FIG. 9). The average diameter of the final afatinib loaded PLGA NPs was 350 nm (PDI=0.234). Zeta potential of both extruded and bare PLGA NPs found to be in between −7 to −5 mV (FIG. 9).

### (12) Synthesis, Physiochemical and Biomimetic Characterization of CAR-T-MNPs

The average diameter of the final cisplatin loaded CAR-T MNPs was 195 nm (PDI=0.117). The zeta potential of both extruded and bare PLGA NPs was found to be in between −7 to −5 mV (FIG. 9B). The size of the CAR-T-MNPs was found to be ˜195 nm, with approximately a 25-nm increase compared to the bare PLGA NPs (˜170 nm) (FIG. 9B). The increase in size from coating is comparable to studies where T cell membrane was used to coat silica nanoparticles to be used as a theragnostic drug delivery system. TEM images of CAR-T-MNPs revealed a core shell structure of the particle with particle size correlating to the DLS data (FIG. 9A). Low polydispersity shows that the particles were homogenous. The loading efficiency of the CAR-T-MNPs was observed to be approximately 60% and the drug release kinetics displayed an initial burst release followed by a sustained release up to 21 days of incubation at 37° ° C. in PBS (pH 7.2) (FIG. 9C).

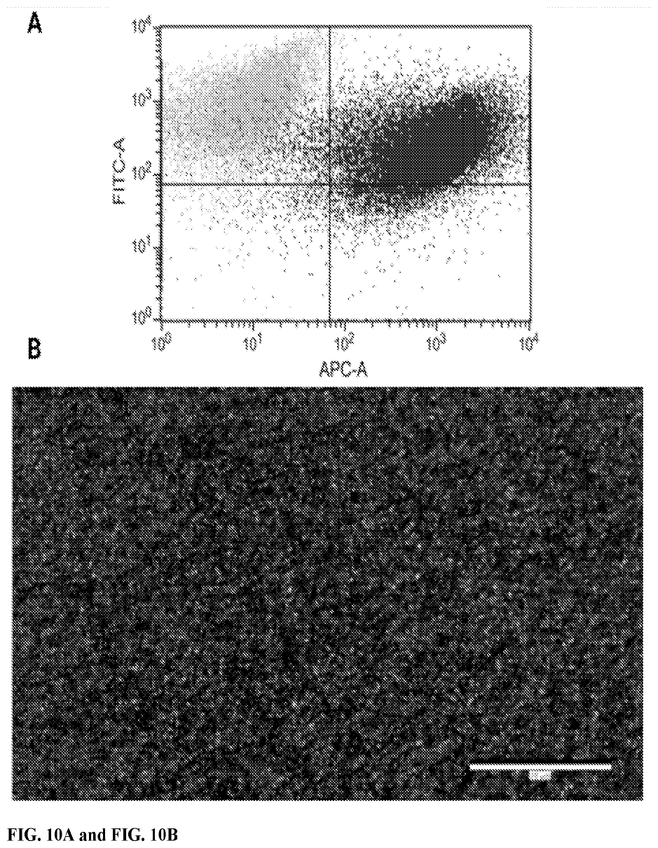

Coumarin-6 loaded PLGA NPs were used for detecting PLGA NPs (FITC channel). After extrusion, membranes were stained with lipophilic DiD dye to detect CAR-T membrane (APC channel) presence on the nanoparticles, using the flow cytometry. Based on the flow cytometric data, the coating efficacy of coumarin-6 PLGA NPs with cell membranes was between 72-77% on both transduced and non-transduced CAR-T-cell membrane extruded groups. Further, coating confirmation of PLGA NPs with CAR-T-cell membranes was assessed via fluorescent microscopy. In the merged channel, yellow-colored particles detected by DiD staining (red) of lipids on CAR-T membrane coating, and coumarin-6 dye (green) from core PLGA NPs confirms the coating of the NPs with membranes (FIG. 10).

Additionally, we performed flow cytometry using an antibody targeting myc tag to confirm the presence of HER2 CAR on the extruded NP surface. The results showed a distinctive shift (55-60% positivity) for CAR-T membrane coated PLGA NPs when compared to that of the non-transduced T-cell membrane NP group (FIG. 9D). In addition, Western Blot analysis of isolated CAR-T membranes also showed bands respective to both CD3 and anti HER2 CAR proteins. Thus, anti HER2 CAR protein presence on isolated jurkat membranes was confirmed (FIG. 9E). Overall, CAR-T-MNP formulation processes were optimized to obtain engineered cell membrane coated NPs.

### (13) CAR-T MNP Membrane Extruded PLGA NP Coating Efficacy Optimization by Flow Cytometry and Fluorescent Imaging

After extrusion of PLGA NPs with isolated membranes, membrane coating efficacy was measured via spectral fluorescent microscopy and flow cytometry. Coumarin-6 loaded PLGA NPs were used for detecting PLGA NPs (FITC channel). After extrusion, membranes were stained with lipophilic DiD dye to detect coating efficacy (APC channel), and CAR-T-MNPs were quantified using the flow cytometry (FIG. 10).

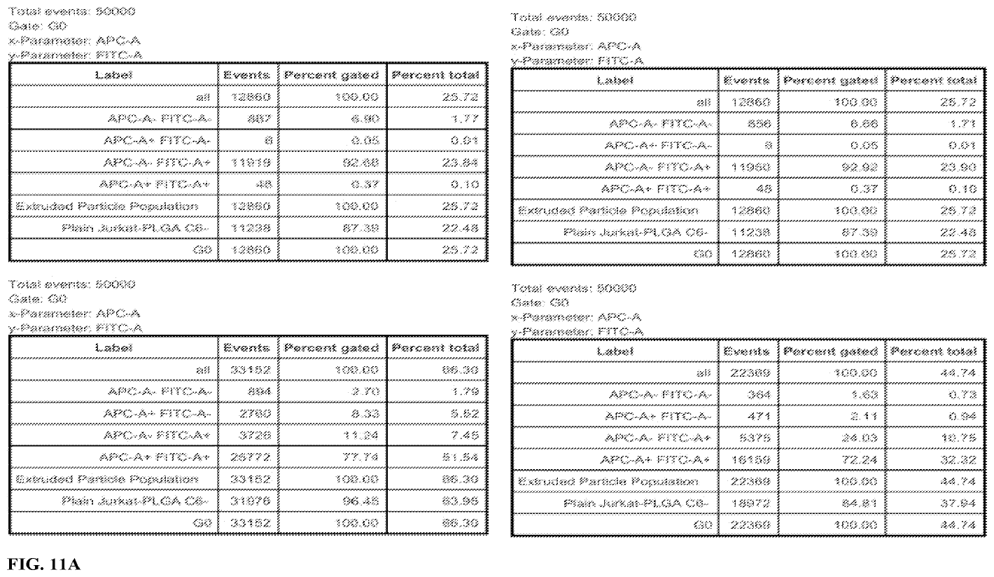

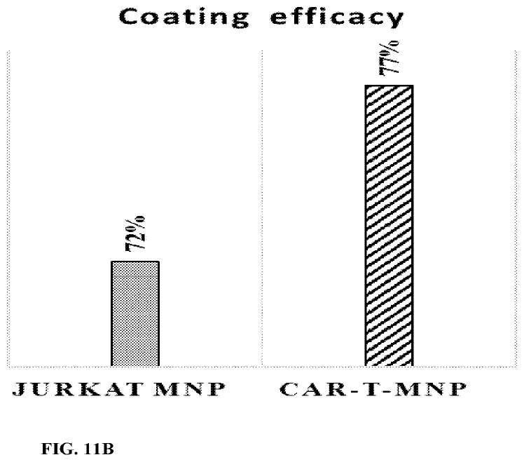

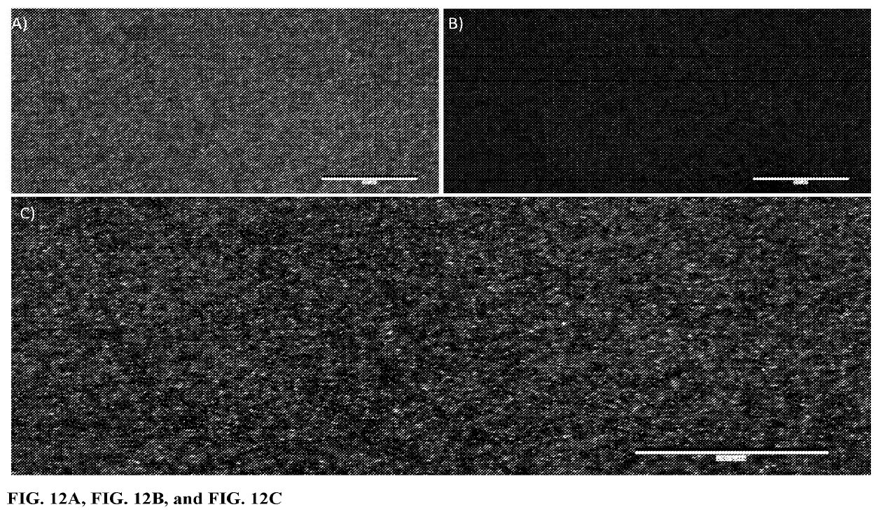

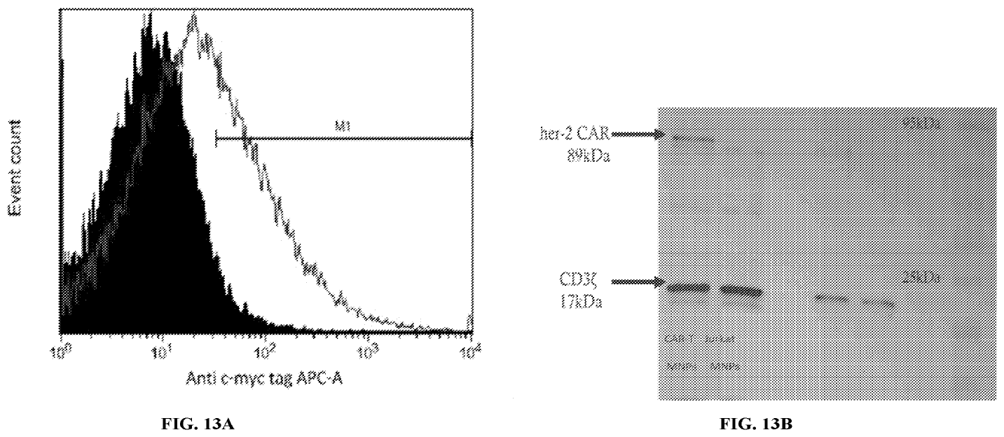

Based on flow cytometry statistic data, coating efficacy of Coumarin-6 PLGA NPs with cell membranes was between 72-77% on both transduced and non-transduced CAR-T-cell membrane extruded groups. For additional confirmation, coating efficacy of CAR-T-cell membranes with PLGA NPs was assessed via spectral fluorescent microscopy (FIG. 11). As shown in FIG. 12, CAR-T-MNPs loaded with Coumarin-6 and stained with DiD were able to be imaged under FITC and red channels. In addition, in the merged channel we obtained yellow looking NP's which confirms the coating of the NPs with membranes.

### (14) Anti HER-2 TCR Existence Confirmation on Extruded NPs

For the confirmation of anti HER-2 CAR receptor of extruded NPs, CAR-T-MNPs were checked on flow cytometry in terms of anti HER-2 CAR existence on their surfaces. Based on the flow cytometry results (FIG. 13A), CAR-T membrane coated PLGA NPs showed a distinctive shift compared to that of the non-transduced T-cell membrane NP group and consisted of 55%-60% anti HER-2 positive NP population. In addition, in FIG. 13B, Western Blot analysis of isolated CAR-T membranes also showed bands respective to both CD3 and anti HER-2 CAR molecules. Thus, engineered anti HER-2 CAR molecule existence on isolated Jurkat membranes has been shown (FIG. 13B).

### (15) Hemocompatibility and Cyto-Compatibility of CAR-T-MNPs

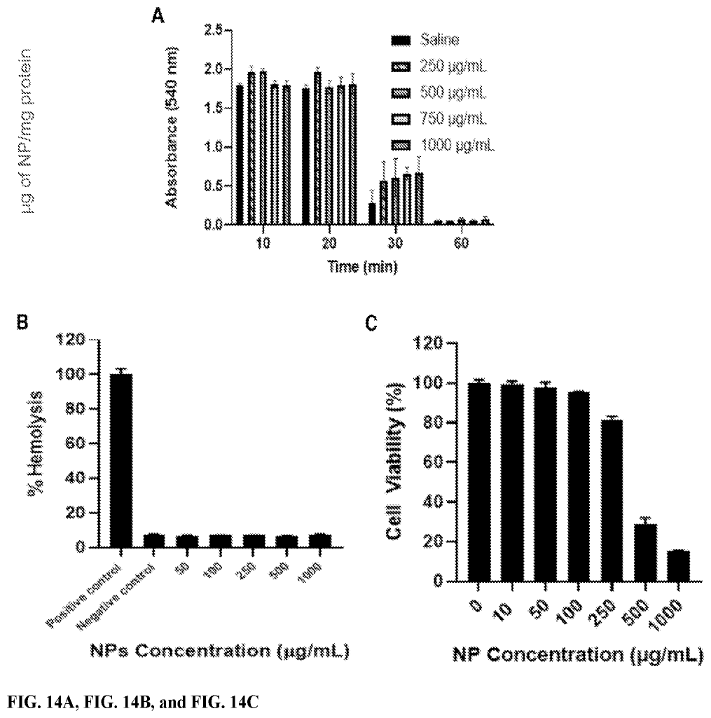

To test the hemocompatibility of CAR-T-MNPs, their hemolysis and blood clotting properties were investigated. The extruded NPs were hemocompatible since CAR-T-MNPs-induced hemolysis was measured <5% even up to 1000 μg/mL concentration of NPs (FIG. 14A). According to the criterion in the ASTM E2524-08 standard, percent hemolysis >5% is considered toxic to red blood cells (ASTM E2524-08, 2013). Thus, CAR-T-MNPs showed complete compatibility for all concentrations. Effects of CAR-T-MNPs on blood clotting characteristics were also tested and found to have no significant effect on the blood clotting cascade up to 1000 μg/mL when compared to the saline control (FIG. 14B).

Cyto-compatibility of CAR-T-MNPs were analyzed by survival study against healthy AT1 cells. Various concentrations from 0-1000 μg/mL were used for 48-hour treatment and analysis. CAR-T-MNPs showed very minimal toxicity for concentrations ≤250 μg/mL. The higher toxicity observed in the higher concentrations are a result of longer exposure in a static 2D model (FIG. 14C).

### (16) In Vitro Uptake Studies of CAR-T-MNPs

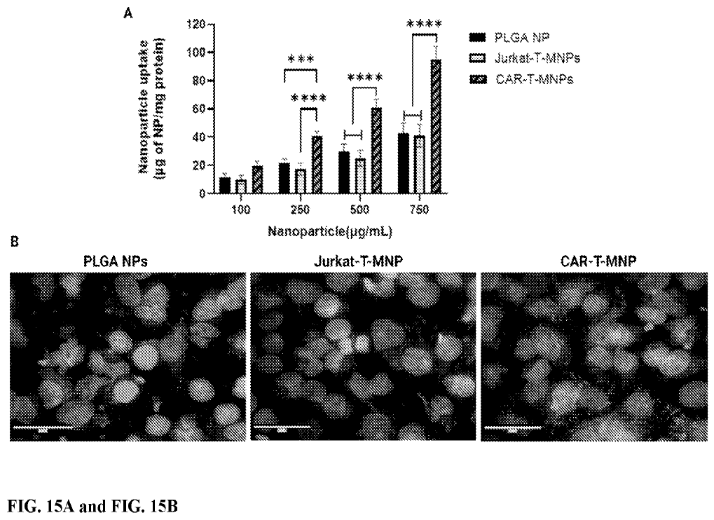

The targeting efficiency of the CAR-T-MNPs were analyzed in-vitro by uptake studies. Briefly HER2 expressing A549 cells were used to measure the CAR-T-MNPs uptake efficiency compared to PLGA NPs and Jurkat-T-MNPs. Coumarin-6 loaded PLGA NPs coated with CAR-T-MNPs or Jurkat-T-MNPs were incubated for 30 minutes with A549 cells. The cells were washed and imaged using microscopy. Microscopy images of cells after incubation (FIG. 15B) indicated that anti-HER2 CAR-T-MNPs had a significantly higher uptake by the A549 lung cancer cells compared to that of the Jurkat-T-MNPs group or PLGA NPs. Spectrophotometric analysis of the NP uptake exposed cell lysates also revealed that CAR-T-MNPs had a significantly higher uptake by A549 cells compared to the other groups (FIG. 15A) for a range of concentrations from (100 L-750 μg/mL). The uptake of CAR-T-MNPs was around twice as much as both the comparison groups for all concentrations above 250 μg/mL. Thus, the correlating microscopy and spectroscopy study proves that the functional anti-HER2 molecule on the CAR-T-MNPs significantly enhanced its cancer targeting and uptake onto lung tumors.

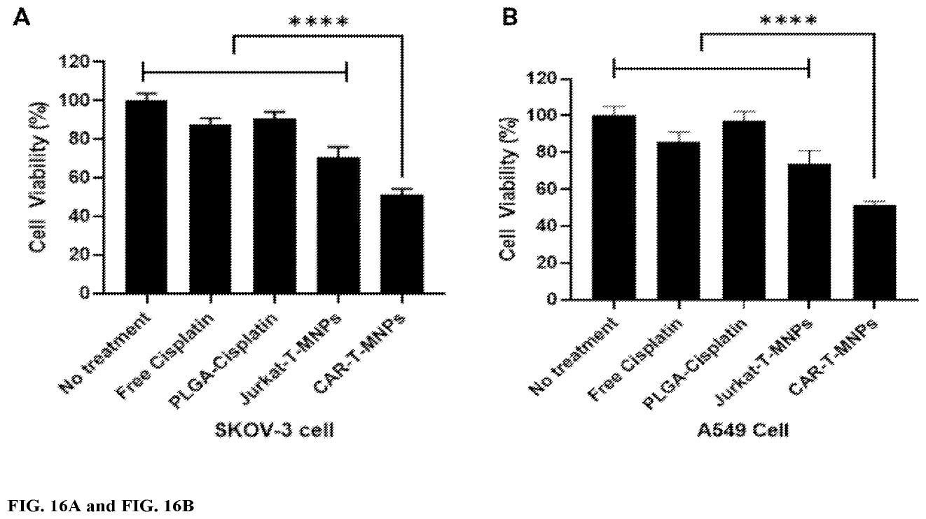

In vitro cancer killing efficacy was determined by cytotoxicity analysis on A549) and SKOV-3 ovarian cancer cell lines. The IC50 (half maximal inhibitory concentration) value of cisplatin on both the cell lines was 7.5 μM and 4 μM respectively. A549 cell line is moderate to high expression of HER2 protein even if it does not contain gene amplification. The cancer killing efficacy of the CAR-T-MNP was also tested by viability assay after 48 hours (FIGS. 16A-B and 17). We observe that the CAR-T-MNP shows significantly higher cancer killing efficiency compared to all the other comparison groups. This result co-related and demonstrates the effect of higher uptake of CAR-T-MNPs on HER2 expressing cells compared to free drug or uncoated NPs. We can also see that Jurkat-T-MNPs have a significantly lower cancer killing efficiency compared to CAR-T-MNPs, this shows NPs transport into the cells are not just passive diffusion or membrane fusion rather an active targeting-based killing. These results indicate that proposed CAR-T membrane coated NPs can be used to improve chemotherapeutic efficacy to effectively treat lung cancer.

### (17) In Vitro Live-Dead Assays

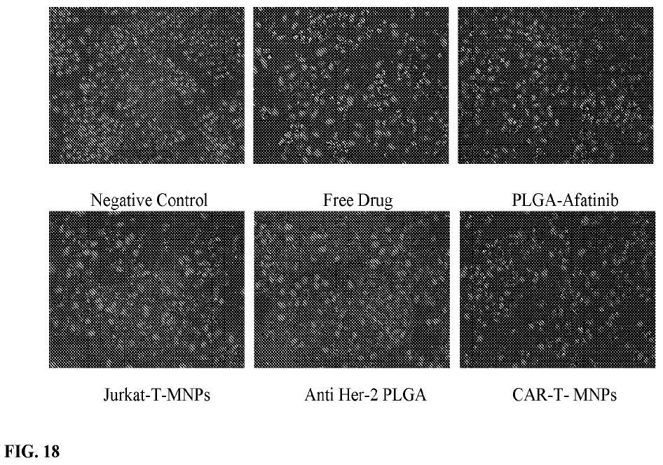

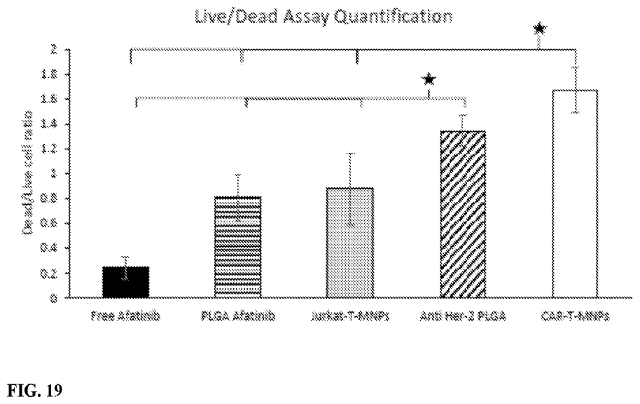

A549 cells were seeded (5×10³ cells/well) in 96-well plates and incubated overnight. As shown in FIG. 18, free afatinib and afatinib-loaded PLGA NPs induced relatively low cell death compared to anti HER-2 PLGA NPs and CAR-T-MNPs. When membrane and antibody groups were compared with each other, Jurkat-T-MNPs and anti HER-2-PLGA NPs loaded with afatinib showed similar cell killing trends but were still lower than that of the CAR-T-MNP group. When image analysis was conducted via image J software on all groups (n=4 for each group), the CAR-T-MNP group showing statistically higher dead/live ratio in exposed cells compared to other groups (FIG. 19). These results indicate that CAR-T-MNPs have superior cell killing ability compared to the bare membrane coated or antibody conjugated nanoparticle groups. This low effect of antibody conjugated nanoparticle group might be due to the wrong orientation via NHS conjugation method on the PLGA NPs, which might alter the interaction of anti HER-2 antibody molecules to the targets and inhibit their binding activity on the cells.

### (18) In Vivo Targeting Efficiency Evaluation of CAR-T-MNPs

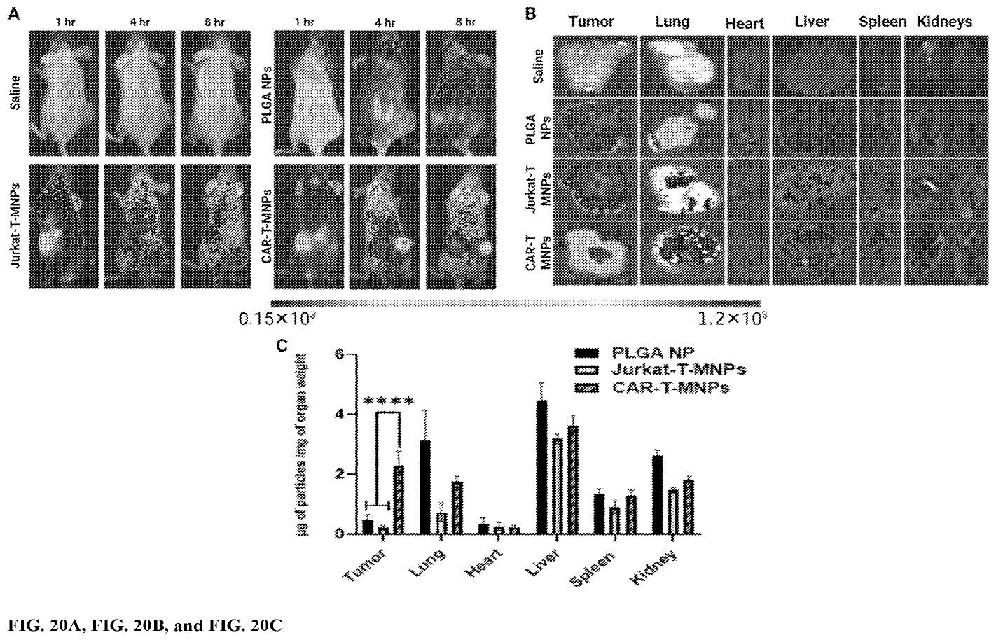

The in-vivo targeting efficiency of the CAR-T-MNPs was analyzed by a biodistribution study on a tumor bearing mice model. PLGA NPs were loaded with ICG dye for deep-tissue imaging and then coated with either anti-HER2 CAR-T-cell or Jurkat cell membranes. To ascertain whether the CAR-T-MNPs can efficiently target, localize and accumulate at tumor sites, their biodistribution in a HER2 expressing tumor xenograft mouse model was assessed (FIG. 20A).

All the groups had NPs accumulated mainly in the liver and spleen in 1 hour. However, PLGA NPs gradually cleared from systemic circulation and from all organs including tumors without any signs of specific targeting towards tumor. On the other hand, CAR-T-MNP groups showed gradual increases of fluorescence in tumors over the course of study, and slowly cleared from the body after 8 hours (FIG. 20A). This increase in accumulation can be attributed to the long circulation times without much clearance because of the several proteins on CAR-T-cell membrane coating. Especially tumor accumulation properties of Cytotoxic lymphocyte T cells is quite evident by our results and it also shows how the membrane proteins play a major role in improving targeting of conventional nanoparticles e.g., PLGA NPs, silica NPs, etc., CAR-T-cell membrane with various proteins such as TCR complexes which can recognize neoantigens from a individuals tumor cells and can improve targeting when used as a platform for personalized medicine in developing this technology. Intensities from the implanted tumors also showed a similar trend to the in vivo images. As shown in the ex vivo organ and tumor images, all groups showed similar accumulation in the kidney and liver, but when comparing the tumor tissue, CAR-T-MNPs showed significantly higher accumulation compared to that of the Jurkat-T-MNPs or PLGA NPs. Spectrophotometric analysis of lysed organs showed a similar trend of both in vivo and ex vivo measurements (FIG. 20B). In the kidney and heart, membrane coated NPs showed lower accumulation compared to the PLGA NP group. In the liver and spleen, despite a trend of low accumulation of membrane coated groups, the amount of particle per mg of organ remained relatively similar in all groups. In the kidney, heart and lungs, membrane coated NPs showed lower accumulation compared to the PLGA NPs & Jurkat-T-MNPs group. Lastly, when tumor accumulation is compared, CAR-T-MNPs had significantly higher accumulation in the tumor tissue (2.3 μg of particle/mg of tumor tissue) compared to that of both the Jurkat-T-MNPs and bare PLGA NPs group (0.21 and 0.46 μg of particle/mg of tumor tissues, respectively) (FIG. 20C). The study therefore shows the significant enhancement of CAR-T-MNPs in tumor targeting and accumulation. The CAR-T-MNPs thus acts as a viable carrier for treatment of HER2 expressing lung tumors.

### (19) In Vivo Therapeutic Efficiency of CAR-T-MNPs

### Tumor Delay Analysis

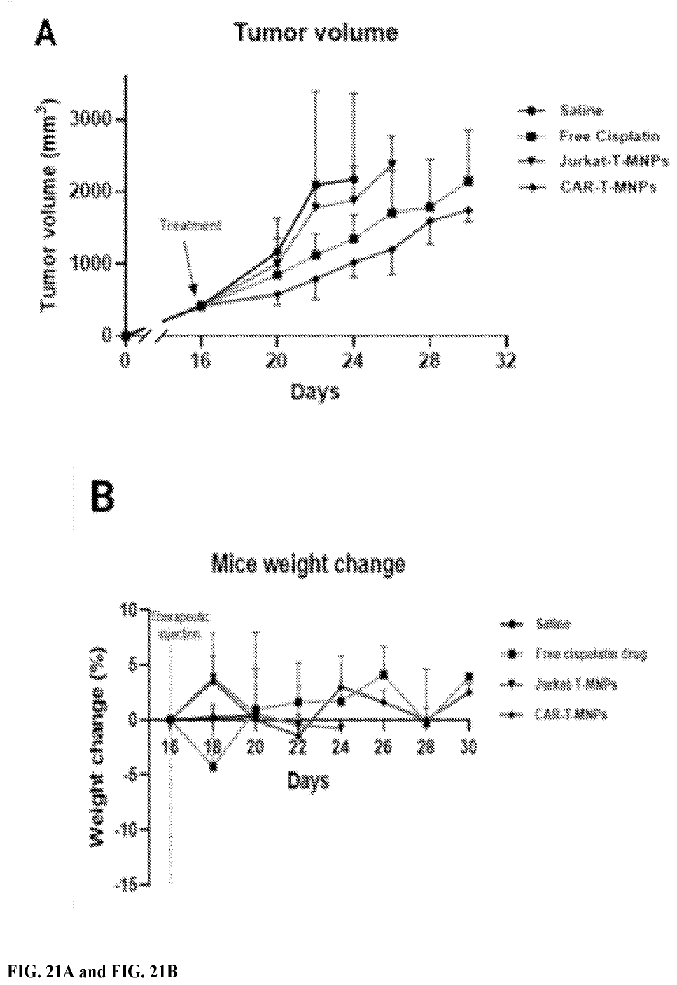

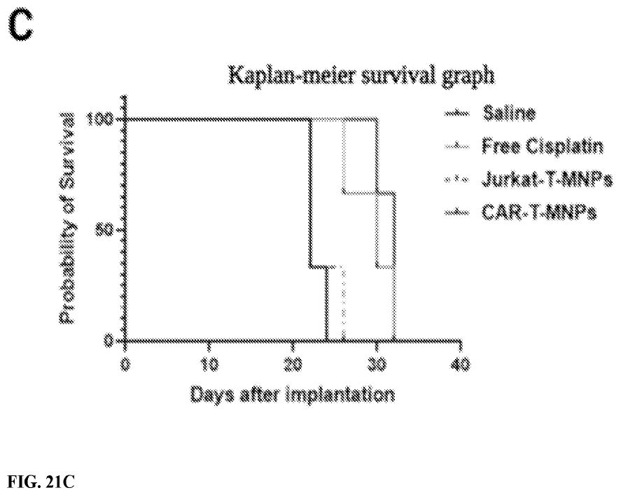

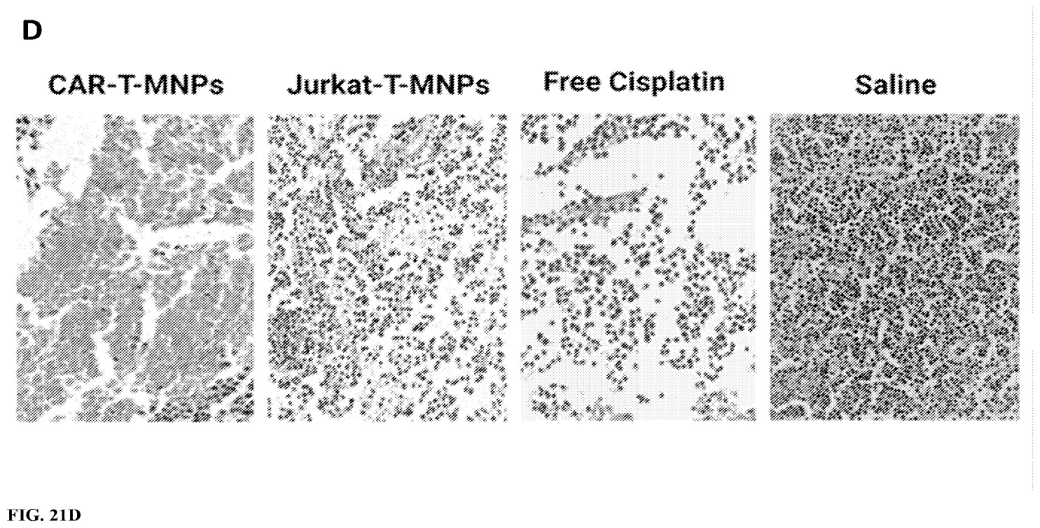

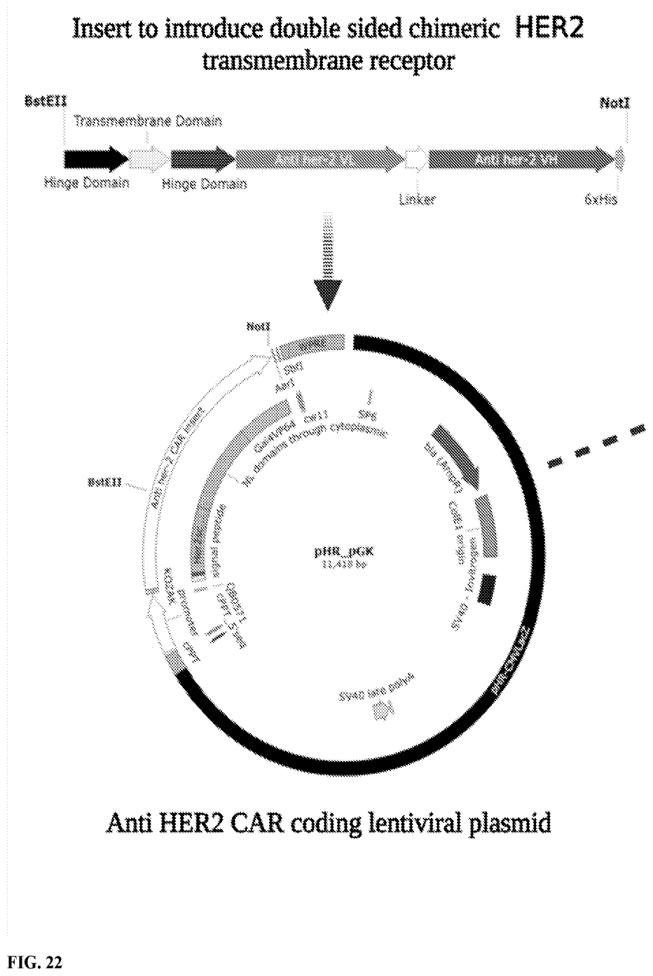

The in vivo therapeutic effect of CAR-T-MNPs was analyzed using a tumor delay/reduction study in A549 tumor bearing nude mice, where the effect of CAR-T-MNPs in delaying the tumor growth was compared to that of Jurkat-T-MNPs and free cisplatin. The cisplatin concentration for all the groups was equal (3 mg/kg). We observed that after 2-week post treatment study, CAR-T-MNPs had significant reduction in the tumor volume compared to that of Jurkat-T-MNPs (FIG. 21A). The tumor volume growth of CAR-T-MNPs was also low compared to that of free cisplatin where we see an increasing trend. We also observed a very high standard deviation for free cisplatin and Jurkat-T-MNPs, unlike CAR-T-MNPs, which exhibited a very consistent reduction through all the animals. Though the objective was to compare tumor delay for a single dose of NPs, the tumor delay trend indicates that with multiple doses, the tumor delay can be greatly improved for CAR-T-MNPs compared to that of the other groups (FIG. 21A). This is also elucidated in the survival of the animals, where we see a significantly higher survival of the CAR-T-MNPs group compared to that of free cisplatin or Jurkat-T-MNPs (FIG. 21C). The improved survival can be implied to reduced accumulation of CAR-T-MNPs in other organs and off-target delivery of drug. Off-target delivery of chemo drugs or un-targeted NPs loaded with chemo drugs can lead to adaptive resistance, organ toxicity and limited efficacy⁵⁹. Along with FDA approved PLGA polymer material and CAR-T-cell membrane coated groups showed no significant weight change of mice, indicating low systemic toxicity of the nanoparticles and signaling good translative potential of our drug delivery platform (FIG. 21C).

### Histology Analysis (H & E Staining)

The histological analysis using H & E staining to tissue slices showed a remarkable decrease in the tumor cells in the CAR-T-MNPs treated group compared to that of other groups and the control60. In vivo toxicity of the CAR-T-MNPs group was also analyzed by H & E staining, and the results indicated minimal systemic toxicity of the CAR-T-MNPs group as compared to other groups. In the liver and spleen, despite a trend of low accumulation of membrane coated groups, the amount of particle per mg of organ remained relatively similar in all groups. Consistently, through histological analysis using hematoxylin and eosin (H & E) staining onto tissue slices, a more marked reduction of tumor cells in CAR-T-MNPs treated tumor was detected than other control groups (FIG. 21D). The H & E staining assay also indicated the minimal systematic toxicity of CAR-T-MNPs with decreased cellularity. There was no obvious organ damage or toxic side effects discovered in any of the groups, however, significant necrosis existed in tumors in the mice of CAR-T-MNPs treated groups (FIG. 21D).

### b) Discussion of Results

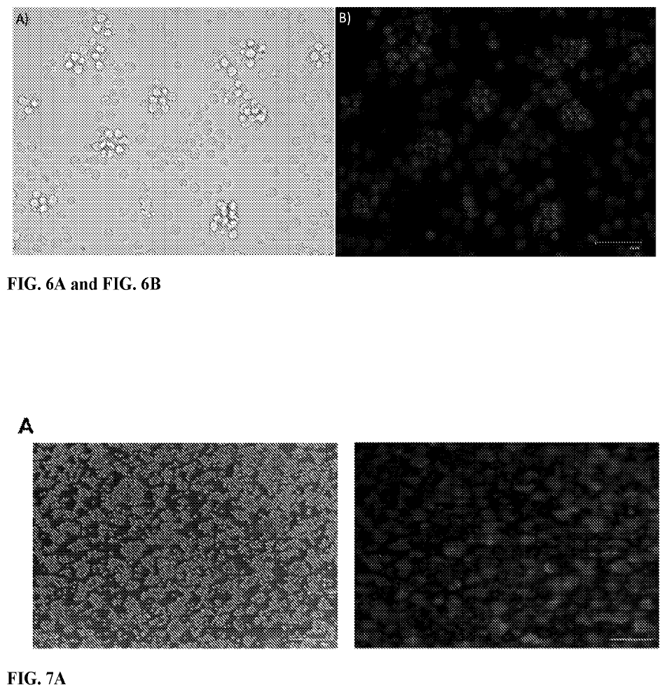

In this research, we have successfully created the anti HER-2 CAR expressing Jurkat cell line via lentiviral transduction. In transduction studies, Lenti-X cell line is used to produce lentiviral particles carrying anti HER-2 CAR gene. Lentivirus titer measured using anti-p24 ELISA kit and found lenti-X cells were able to produce 10⁸ lentiviral titer (TU). Based on lentiviral transduction results and optimization studies (FIG. 5), 10 multiplicity of infection (MOI) was chosen to transduce 105 Jurkat cells. After transduction, we observed red fluorescent cells in the culture after 48 hours (FIG. 6). Flow cytometric analyses showed that cells were able to achieve stable expression of both mCherry and anti HER-2 CAR receptors over 90% (FIG. 7).

In the scope of the proposal, anti HER-2 CAR-T-cells were fabricated and characterized, and then anti HER-2 CAR-T-cell membrane isolation and extrusion with PLGA NPs to formulate CAR-T-MNPs were performed. The zeta potential of the CAR-T-MNPs was approximately −7.2 mV. The size of the CAR-T-MNPs was monitored using dynamic light scattering and was found to be 330 nm, with approximately a 20-nm increase compared to the Bare PLGA NPs (130 nm) (FIG. 9). In addition, in the literature researchers found membrane coated PLGA nanoparticles were stable up to 15 days. The loading efficiency of the CAR-T-MNPs was observed to be approximately 48% and the drug release kinetics displayed an initial burst release followed by a sustained release up to 21 days of incubation at 37° C. in PBS (pH 7.2). The rate of release kinetics differed across bare PLGA and the CAR-T-MNPs (FIG. 8A). This result might be due to the diffusion barrier effect of the membrane layer. Additional coating of cell membranes on the PLGA NPs might either have slowed down the degradation process of the PLGA NPs or drug diffusion to the surrounding. CAR-T-MNPs were tested for blood 25 clotting and hemolytic properties. CAR-T-MNPs-induced blood hemolysis was observed to be <1% up to 1000 μg/mL (FIG. 8C). Effects of CAR-T-MNPs on blood clotting characteristics were also tested and found no significant effect on the blood clotting cascade up to 1000 μg/mL when compared to the saline control (FIG. 8B). In addition, TEM images revealed that membrane coated NPs had shown as core shell or onion like structures, which confirms the membrane coating on the surface of PLGA NPs (FIGS. 9A,9B, 10A, and 10B). To identify the specific anti HER-2 CAR receptor in the CAR-T-MNPs formulations, the particles were stained with anti c-myc tag antibody to detect ScFv on the particle surface. The samples were analyzed using a flow cytometer without SSC threshold. Compared to the unstained control, stained T-MNP samples displayed a significant mean fluorescence intensity shift (˜%70) (FIG. 12). Therefore, CAR-T-MNP formulation processes were optimized to obtain engineered cell membrane coated nanoparticles, and CAR-T-MNPs were characterized in terms of coating efficacy, ligand existence on the surface, drug loading/release and hemocompatibility.

In addition, anti HER-2 antibody purification, optimization and conjugation to PLGA NPs had been conducted as a comparison group for our experiments. Indirect ELISA results of anti her-2 PLGA NP conjugation revealed that we have achieved ˜%70 conjugation efficacy (FIG. 9A). On the other hand, Western Blot analysis showed two distinctive bands respective to heavy and light chain of the antibodies (FIG. 9B). Interestingly, we have noticed two consecutive bands of both chains of antibodies. We estimate that those bands belong to PLGA NPs conjugated antibody chains which they dragged behind the free chains during the SDS-PAGE electrophoresis. Competitive ELISA confirmed that conjugated antibodies were able to compete with free anti her-2 antibodies and bind to its target (HER-2 protein) between the concentrations of 1-100 μg/ml (FIG. 9C).

A549 cell lines were utilized to examine the CAR-T-MNPs uptake effect in comparison with anti HER-2 PLGA NPs and Jurkat-T-MNPs (FIG. 15). All nanoparticles were tested at different concentrations (100 μg/mL-750 μg/mL). The uptake of CAR-T-MNPs was significantly higher than the Jurkat-T-MNPs (FIG. 15) in all concentrations. The cellular uptake of the CAR-T-MNPs was increased with increasing concentration. In addition, uptake of CAR-T-MNPs was significantly higher than anti HER-2 PLGA NPs in moderate concentrations (250 ug/mL and 500 μg/mL). This is due to the static uptake conditions and saturation of the cell samples with targeting molecules. Due to the enhanced uptake of CAR-T-MNPs, it was proposed that the CAR-T-MNPs can present functional anti HER-2 molecules on their surface that are contributing to the NP uptake significantly.

The cancer cell killing properties of these CAR-T-MNPs were investigated; we found them to be effective against A549, SKOV and MDA-MB-231 cell lines in vitro. Before the cancer cell killing studies, flow cytometry studies verified the presence of HER-2 antigen on the A549 cancer cells. It has been shown that HER-2 expression levels of various N-SCLC cell lines via different methods and found A549 cell line is moderate to high expression of HER-2 protein even if it does not contain gene amplification. Furthermore, the group also provided evidence of anti-HER-2 antibody binding and the synergistic effect of trastuzumab and different chemotherapeutic drugs on the A549 cell line. Similar to the above-mentioned literature, A549 cells stained with purified anti HER-2 antibodies showed a moderate to high shift (˜%75 to %90) in the flow cytometry histogram. In addition, HER-2 overexpressing SKOV-3 and HER-2 negative MDA-MB-231 cell line were also checked for their expression levels and it was been found that SKOV-3 cells showed a distinctive shift, whereas MDA-MB-231 did not show a significant shift in the flow cytometric histogram in terms of HER-2 binding (PE channel).

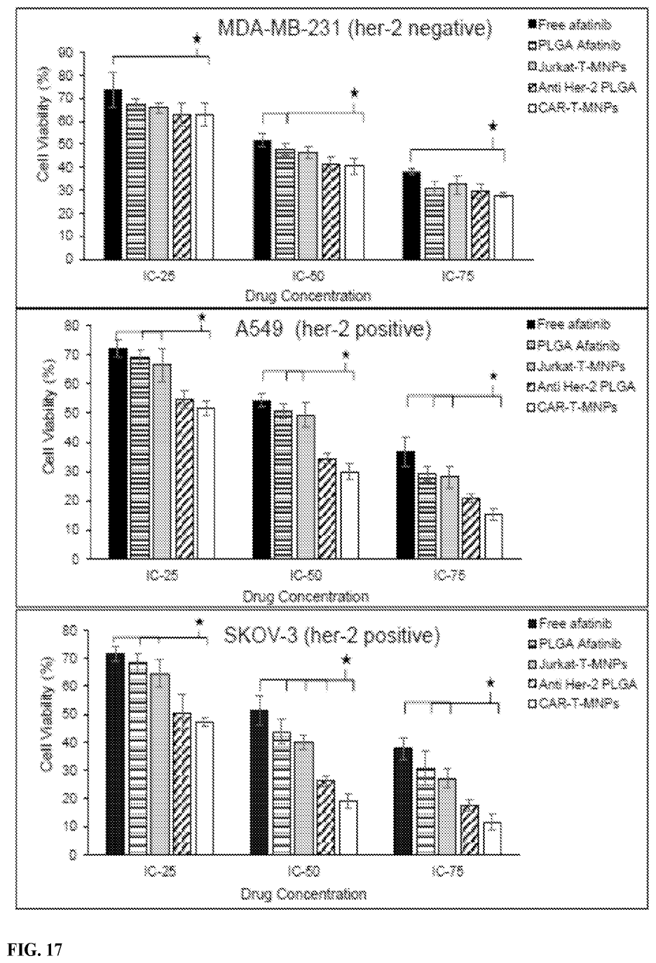

In the literature, inhibitor concentration at 50% cell viability (IC-50) for afatinib against different cell lines has been described. To verify the IC50, a pharmacokinetics study on afatinib was carried out by exposing the SKOV-3, A549 and MDA-MB-231 cell lines to varying afatinib concentrations over a 48 hour period. IC50 values of afatinib for SKOV-3, A549 and MDA-MB-231 were found to be approximately 1.4 μM, 4.3 μM, and 3.7 μM, respectively. Following, to test the therapeutic efficiency of CAR-T-MNPs on SKOV-3, A549 and MDA-MB-231 cell lines, the cells were incubated with afatinib-loaded PLGA-NPs, Jurkat-T-MNPs, anti her-2 PLGA, CAR-T-MNPs and free afatinib for 48 hours at IC25, IC50 and IC75 drug concentrations (FIGS. 16 and 17).

For cancer cell killing studies, Jurkat-T-MNPs served as membrane-coated negative controls. PLGA NPs served as a non-membrane coated negative control. A free drug group was also included to control against nanoparticle delivery systems. Both anti HER-2 PLGA and CAR-T-MNPs were found to be significantly more efficient than any of the negative control groups, including the free afatinib at all concentrations on high HER-2 expressing cell lines (SKOV-3 and A549) (FIG. 17). On the other hand, on low HER-2 expressing cell line MDA-MB 231, anti-HER-2 PLGA and CAR-T-MNPs showed significant efficiency only in higher concentrations (IC50 and IC75) compared to free afatinib only (FIG. 17). This effect might be the result of a low available number of HER-2 protein on the surface of the cells, leading to lower uptake and killing efficacy compared to higher HER-2 expressing cell lines. When CAR-T-MNPs compared to anti HER-2 PLGA NPs, membrane coated NPs showed significant higher killing efficacy on SKOV-3 and A549 cell lines at higher concentrations (IC50 & IC75) (FIG. 17). It is estimated that the non-significant results on lower concentrations might be due to the relatively low NP uptake number. Additionally, significantly higher killing efficacy of CAR-T-MNPs might have resulted from a higher number of uptake due to either a higher number of copies on the surface of CAR-T-MNPs, or the correct orientation (ScFv towards to the target molecule) of the anti HER-2 molecules on the NP surface, or other surface protein interactions might serve as synergistic enhanced binding to target cells. As a second confirmation of the cell killing efficacy of CAR-T-MNPs, live/dead fluorescent cell killing assays were performed and revealed parallel results with MTT assays (FIGS. 18 and 19). Based on the live/dead cell killing assay, both anti HER-2 PLGA NPs and CAR-T-MNPs showed significant dead/live cell ratios (˜1.3 and 1.6, respectively) compared to negative controls (FIG. 18).

The biodistribution study examined whether intravenously injected CAR-T-MNPs targeted subcutaneous tumors in the tumor implanted mice. A549 cells were injected into NGC mice at 2×10⁶ cells/mouse via subcutaneously to develop a lung cancer model. All the particles were accumulated mainly in the liver and spleen in 1 hour. However, PLGA NPs gradually cleared from systemic circulation and from all organs including tumors. On the other hand, anti HER-2-PLGA and CAR-T-MNP groups showed gradual increases of fluorescence in tumors over the course of study and slightly started to be cleared from the system after 8 hours (FIG. 20). Additionally, CAR-NPs showed significantly higher accumulation on the tumor regions compared to anti HER-2 PLGA NPs (FIG. 20). At the end time point, all animals were autopsied and ex vivo organ images taken to be analyzed. First, the imaging system was not able to pick fluorescent data from the spleen and heart. This might be due to a relatively small concentration or the clearance of the drug in these organs at that time point. Significant accumulation of the nanoparticles in lung tissues of both PLGA and anti HER-2 PLGA NP groups have also been observed. On the other hand, CAR-T-MNPs and anti HER-2 PLGA NP groups showed relatively higher accumulation of NPs in the tumor tissues (FIG. 20A).

Spectrophotometric analysis of homogenized organs showed a similar trend of both in vivo and ex vivo imaging measurements (FIG. 20B). In the liver and spleen, despite a trend of low accumulation of membrane coated groups, the amount of particle per mg of organ remained relatively similar in all groups. In the kidney, heart and lungs, membrane coated NPs showed lower accumulation compared to the anti HER-2 PLGA NP group. Lastly, when tumor accumulation is compared, CAR-T-MNPs had significantly higher accumulation in the tumor tissue compared to that of the anti HER-2 PLGA NP group (2.3 and 1.4 μg of particle/mg of tumor tissues, respectively).

### c) Conclusions

In conclusion, we have successfully engineered and characterized Jurkat T lymphoma cells with chimeric antigenic receptor, and anti HER-2 CAR engineered T-cell membrane coated afatinib-PLGA nanoparticles were successfully synthesized. These CAR-T-MNP membrane coated NPs displayed a high drug encapsulations efficiency of 61% and released the drug in a sustained fashion over a period of 21 days. The drug release profile was found to be dependent on the existence of membrane on the particles in which the membrane coated nanoparticles provided a slower release. It might be due to the barrier effect of the additional membrane layer. Fabricated CAR T-MNPs were characterized in terms of size (<400 nm), cell combability (up to 1000 μg/mL), stability and hemocompatibility (up to 500 μg/mL).

Moreover, cellular uptake studies showed that CAR-T-MNPs demonstrated superior uptake kinetics compared to bare particles and anti HER-2 conjugated synthetic PLGA NPs. With the help of the anti HER-2 CAR receptor, particles showed selective uptake by her-2 positive lung cancer cells when compared to anti HER-2 negative Jurkat membrane coated PLGA and chemically conjugated anti HER-2 PLGA NPs. These findings also correlated with enhanced therapeutic efficiencies of CAR-T-MNPs. Their cancer killing efficiencies agreed with uptake characteristics. However, detailed uptake binding kinetics of the ScFv's and antibodies is necessary for quantitative assessment and comparison of both systems such as targeting molecule per carrier conjugation direction to the surface and others. Therefore, CAR engineered membrane coated NPs system is a promising cell mimicking drug carrier that can improve the therapeutic outcomes of non-small lung cancer treatments.

### d) Materials and Methods

### (1) Cell Lines and Culture Conditions

Lentiviral packaging cell line Lenti-X 293 T was purchased from Takara-Clontech. Alveolar Type 1 (AT1) cells, HEK 293 T cells, HER2 positive tumor lines A549, SKOV-3 and Jurkat E6-1 were purchased through American Type Culture Collection (ATCC, Manassas, VA). HEK 283 T, A549, and SKOV-3 cell lines were cultured in DMEM medium supplemented with 10% heat inactivated FBS (Corning, NY), 100 U/ml penicillin, and 100 μg/ml streptomycin (Invitrogen, Waltham, MA). ATI cells were cultured in IMDM Medium supplemented with 10% heat inactivated FBS, 100 U/ml penicillin. Lymphocytes and Jurkat cells were cultured in GT-T551 medium (Takara, California), supplemented with 10% FBS and 50 IU/ml IL-2 (Miltenyi Biotec, Cambridge, MA) at 37° C. and 5% CO2.

### (2) Materials

Poly (lactic-co-glycolic acid) ((PLGA), LG 50:50, (Mₙ 25,000-35,000 Da)), LG 50:50 (Mₙ 25,000-35,000 Da)) were purchased from Akina Inc (West Lafayette, IN). Polyvinyl alcohol (PVA, MW 15,000-25,000), NHS, bovine serum albumin (BSA), Dulbecco's Modified Eagle's Medium (DMEM), Iscove's Modified Dulbecco's Medium (IMDM), Protease inhibitor cocktail, EDC (1-ethyl-3-(3-dimethylaminopropyl)-carbodiimide) and Triton R X-100 were purchased from Sigma-Aldrich (St. Louis, MS), and dichloromethane from Merck (Kenilworth, NJ). Cisplatin was purchased from Cayman chemicals (Ann Arbor, MI). SDS-PAGE gel, and Mini PVDF transfer pack were obtained from Bio-Rad. The Mini Extruder Kit was purchased from Avanti Polar Lipids (Alabaster, AL). Formvar coated copper TEM grids were purchased from Electron Microscopy Sciences (Hatfield, PA). Fetal bovine serum (FBS), 1× trypsin EDTA and penicillin-streptomycin were ordered from Invitrogen. All reagents were of analytical grade.

### (3) Preparation of PLGA Nanoparticles

Briefly, Afatinib (10 mg) was added dropwise to 3 mL of dichloromethane containing PLGA (90 mg) sonicated for 3 minutes and resultant emulsification was added dropwise to 20 mL of aqueous phase containing 5% poly(vinyl alcohol) and emulsified using ultrasonication for 10 minutes at an 1.30 on 0.30 off. The mixture was subsequently stirred overnight in the fume hood at room temperature to evaporate organic solvents. The nanoparticles were washed three times with ultra-pure water using ultracentrifugation at 15.000 rpm 15 min and freeze-dried (−57° C., 0.090 mbar, 24 h, Labconco). For in vitro and in vivo particle tracking purposes, 10 μg of coumarin-6 green dyes or ICG infrared dyes was added to the PLGA dichloromethane solution prior to PLGA nanoparticle synthesis (without drug/water phase). To determine loading efficacy, afatinib encapsulated in the PLGA was measured using UV-VIS Spectrophotometer.

Cisplatin loaded PLGA nanoparticles (MNPs) were synthesized using modified version of standard double emulsion protocol. Briefly, 10 mg Cisplatin dissolved in 1 ml of dimethyl formamide was added dropwise to 3 ml of dimethyl formamide (DMF) containing 100 mg of PLGA and sonicated for 2 minutes. Resultant emulsification was added dropwise to 20 ml of aqueous phase containing 5% poly (vinyl alcohol) and emulsified using ultrasonication for 5 minutes. The mixture was subsequently stirred, centrifuged at 20,000 RPM for 20 mins. The NPs were washed, collected, and freeze-dried. For in vitro and in vivo uptake and biodistribution studies, 5 μg of coumarin-6 green dyes or ICG infrared dyes was added to the PLGA DMF solution instead of cisplatin during PLGA nanoparticle synthesis.

### (4) Hybridoma Cell Culture, Purification and Characterization of 4D5 Anti HER-2 Antibodies

4D5 hybridoma cells were thawed rapidly, then transferred to 5 ml warm RPMI 1640 medium containing 10% FBS and 1% P/S (Gibco) and plated onto 25 cm tissue culture flasks. Cells split 1:5 every 3 days to keep the cell density 10⁶ cells/ml. Split cells into larger plates every-time by adding cells to fresh 10 ml medium in 75 cm2 flasks and 25 ml in 175 cm2 flasks. Gradually, the FBS amount decreased in hybridoma culture from 10% to 0%, and all supernatant was maintained in 4 degrees for purification. For antibody concentration, all the cells collected and spun down at 3000 rpm for 5 minutes at 4° C. Collected supernatant was filtered through 0.22 μm filter and concentrated using Amicon (Millipore). Cells were stirred via ultracel ultrafiltration disc (Millipore 100 KDA cutoff membrane). For purification step, 1 ml HiTrap Protein G HP column (GE healthcare 17-0404-03) was equilibrated via filtering 10 ml pH PBS. Concentrated supernatant was mixed 1:1 with binding buffer pH7.2 and applied to column at 1 ml/min. Column was washed with 20 ml PBS, and antibodies were eluted with 5 ml 100 mM Glycine pH 3. 0.5 ml fractions were collected into tubes containing 50 μL 0.5M Tris (pH 9.5) and gently mixed to neutralize pH. Protein contents in purified fractions were measured via nanodrop (280 Abs), and functionality of the antibodies was tested via flow cytometer on A549 cells. Fractions containing high concentrations of antibody were kept at 4° C. until used.

### (5) Lentiviral Vector Production

pHR_PGK_antiHer-2_synNotch_Gal4VP64 lentiviral plasmid (used as backbone for expressing anti her-2 specific CAR plasmids) and pHR_Gal4UAS_tBFP_PGK_mCherry (used as fluorescent reporter for transduction) lentiviral plasmids were a gift from Wendell Lim (Addgene plasmid #85423). Pantropic VSV-G pseudotyped lentivirus was produced via transfection of Lenti-X 293T cells (Clontech #11131D) with anti her-2 CAR plasmids and/or pHR_Gal4UAS_tBFP_PGK_mCherry expression vectors and the viral packaging plasmids psPAX2 and pLP-VSVG using Lipofectamine 3000 (Invitrogen). Supernatants containing lentivirus were collected 48 and 72 hours later.

### (6) Lentiviral Transduction of Jurkat T-Cells

For transduction of Jurkat T-cells, Jurkat T-cells were cultured in a 6 well plate. CD4+ Jurkat cells (500000 cells per well) were spinoculated (2000 rpm 90 minutes/37° C.) and co-cultured for 24 hours with lentiviral supernatants in the presence of 8 μg polybrene (Merck). The cells were cultured for 3 day in 10% FBS media. T-cell surface markers were detected via anti-human CD3, CD4 antibodies (Biolegend) using flow cytometry methods. Her-2 specific CAR expression was detected by Anti-Myc Tag-Alexa 647 (Cell Signaling). Fluorescence response analysis and sorting of live cells were performed using a BD LSR II & BD melody flow cytometer and analyzed with FACSlyzer software (Becton Dickinson).

### (7) CAR-T Cell Generation Membrane Isolation

CAR-T cells expressing HER2 ScFv were created by transduction using lentiviral particles. Cells (10⁸) were washed three times with cold PBS (1×). Before the last spin, cells were counted and resuspended in cold hypotonic buffer Tris-HCl (10 mM, pH7.5) in 10 mL. Protease and phosphatase inhibitor cocktail (PIC) was added (5 μL/million cells) and incubated at 4° C. for 20 minutes. The mixture was centrifuged at 4000 g for 10 minutes. The resulting pellet was resuspended in cold PBS 0.25×+PIC at 1 ml/10 million cells and incubated for another 20 minutes at 4° C. The mixture was centrifuged at 4000 g for 10 minutes, then the pellet was resuspended in cold PBS 1×+PIC and kept on ice. DNAse reaction was performed on the samples based on their DNA contents (for 20 million cells: 20 IU DNAse for 4 hours). After using DNAse, mixture was centrifuged at 4000 g for 10 minutes. The isolated membranes were collected, lyophilized overnight and stored at 4° C. Right before use, membrane lyophilates were weighed and resuspended in a PBS solution.

(8) Coating PLGA Nanoparticles with CAR-T Membranes and Nanoparticle Characterization

CAR-T membrane-derived nanovesicles were prepared as follows; drug loaded PLGA nanoparticles were added via hydrating these NPs with freeze-dried CAR-T membranes, whereas the CAR-T membrane amount was slightly more than the calculated amount of lipid molecule derived from cells to cover all amount of PLGA nanoparticle surface. The mixture was sonicated for 5 minutes using a probe sonicator. Subsequently, the mixture was co-extruded using an Avanti mini extruder. The size distribution and polydispersity of these particles were measured via dynamic light scattering. The in vitro release profile of Afatinib from the CAR-T-MNPs was performed using dialysis method and UV-VIS Spectrophotometer.

### (9) Physicochemical Characterization of CAR-T-MNPs

Dynamic Light Scattering (DLS) technique was used to measure the physiochemical characterizations including size, zeta potential and polydispersity index. To measure size of the nanoparticles, NP suspension (10 μL of 500 μg/mL) was added to 3 mL of deionized water and measured by the DLS in a compatible cuvette. The surface morphology of the formulated nanoparticles was visualized by transmission electron microscopy (TEM). Briefly, freeze-dried nanoparticle samples in DI water were fixed onto ozone treated copper grids (Electron microscopy sciences) and stained with uranyl acetate (0.5%) (Sigma Aldrich). A H-7500 TEM (Hitachi) transmission electron microscope was used to visualize the particle morphologies.

### (10) Characterization of CAR-T Membrane Coating

After extrusion, coating efficiency were measured. Briefly, coumarin-6 loaded CAR-T-MNPs were stained with lipophilic DiD dye and analyzed by flow cytometry (APC channel). To identify the specific anti-HER2 CAR receptor in the CAR-T-MNPs formulations, the particles were stained with anti c-myc tag antibody to detect ScFv on the particle surface. 30

Western Blot analysis was done to confirm the successful transduction of anti-HER2 CAR molecule on isolated membranes. In Western Blot analysis, isolated membranes were run on SDS-PAGE gels under denaturing conditions. Samples were mixed with laemeli buffer containing 10% β-mercaptoethanol, 4% SDS, 125 mM Tris-HCl, 20% glycerol, 0.004% bromophenol blue and run-on gel; then transferred onto nitrocellulose membranes. First membranes were incubated with primary antibody against myc tag to verify HER2 CAR. Anti-human CD3 (heavy chain light chain) were used as housekeeping protein. Blots were visualized using the ECL (Millipore) with ChemiDoc Imager (Bio-Rad). Non-transfected cell membrane coated NPs were used as a control.

(11) Conjugation of Anti HER-2 Antibodies with PLGA NPs

To covalently attach mAbs onto the nanoparticle surface, NHS conjugation chemistry was employed. Right after synthesis of PLGA NPs (20% PLGA-NHS and 80% PLGA), washed particles were added to 1 ml of a 0.3 M MES buffer. Different weight ratios of 1 mg nanoparticles to 5, 50 and 100 μg of mAb were used in conjugation experiments. The reaction mixture was stirred gently for 3 hours at room temperature. Excess linking reagents and soluble by-products were separated by centrifugation at 13,200 rpm for 10 minutes, and the pellet was washed three times with 1 ml PBS, pH 7.4. Finally, mAb conjugated nanoparticles were re-dispersed in 300 μl of PBS, and the protein content was determined by ELISA and nanodrop (280 nm Abs).

### (12) Indirect ELISA Assay

Microtiter ELISA well plates were coated with 100 μl of the HER-2 antigen solution at a concentration of 1 μg/ml in coating buffer. Samples were incubated overnight at 4° C. After incubation time, they were washed with 300 μl wash buffer three times and blocked with 300 μl blocking buffer at 37° C. for 1.5 hours. Plates were washed 3 times and incubated 1.5 hour at 37° C. with different concentrations of unconjugated PLGA, anti HER-2 antibody conjugated PLGA and with standard anti HER-2 antibodies alone. After repeating the washing step, 100 μl of HRP-conjugated secondary antibody (anti-mouse H+L chain, 0.1 μg/ml in dilution buffer) was added to each well and incubated for 1 hour at 37° C. After washing 3 times with the washing buffer, 100 μl TMB substrate solution was added to each well and incubated at room temperature for 30 minutes after adding stop solution to each well. That was followed by the measurement of absorbance values at 450 nm wavelength (Tecan).

### (13) Western Blot

Western Blot analysis was done to confirm the conjugation of anti HER-2 antibody on PLGA NPs. In Western Blot analysis, antibody conjugated nanoparticles were run on SDS-PAGE gels under denaturing conditions. Samples were mixed with laemmli buffer containing 10% β-mercaptoethanol, 4% SDS, 125 mM Tris-HCl, 20% Glycerol, 0.004% bromophenol blue and run on gel then transferred onto nitrocellulose membranes. Proteins/membranes were analyzed by western blotting. Membranes were incubated with primary antibodies against anti-mouse IgG (H+L chain) (Jackson laboratories). Blots were visualized using the ECL (Millipore) with ChemiDoc Imager (Bio-Rad). Non-conjugated nanoparticles were used as a control.

### (14) Particle Size and Zeta Potential Analysis

Freeze-dried nanoparticles were dispersed in deionized water. Their mean particle diameter and the width of the particle distribution (polydispersity index) were determined by photon correlation spectroscopy or dynamic light scattering (DLS) using a Nanobrook 90 plus PALS (BrookHaven Instruments). The particle charge was quantified as zeta potential by Nanobrook 90 plus PALS zetasizer using the Nanobrook 90 plus. All measurements were made in triplicate.

### (15) Surface Morphology

The surface morphology of the formulated nanoparticles was visualized by transmission electron microscopy (TEM). Before observation, freeze-dried nanoparticle samples in DI water were fixed onto ozone treated copper grids and stained with uranyl acetate (0.5%). A H-7500 TEM (Hitachi) transmission electron microscope was used to visualize the particle morphologies.

### (16) Drug Loading Efficacy and In Vitro Drug Release Profile

To determine loading efficacy, cisplatin encapsulated in the PLGA was measured using a UV-VIS Spectrophotometer incorporating the ortho-phenylene diamine technique⁶³. The supernatant solution obtained after ultracentrifugation of NPs during the formulation process was used for loading efficiency determination. For the drug release studies, suspensions of cisplatin loaded NPs in PBS (pH 7.2) were added to dialysis bags (Spectrum Laboratories Inc.) with a molecular weight cut-off (MWCO) of 8000 Da. Dialysis against PBS was carried out at 37° C. up to 21 days. At pre-determined time points, dialysate was collected and stored at −20° C. for analysis. As described by Mohit et. Al.⁶³ dilutions of sample were made using 1 ml of 1.4 mg/ml of OPDA solution (Sigma Aldrich) and 2 mL of phosphate buffer pH 6.8 and heated at 100° C. for 10 minutes to get a light-green color solution. The solution was cooled, and the volume was adjusted to 10 ml by DMF. The concentration of cisplatin was then measured using an absorbance spectrometer at λ706 nm. A standard curve was plotted by measuring the absorbance of known concentrations of cisplatin. The amount of cisplatin released was then determined against the standard curve and was correlated to the cisplatin loading efficiency, which was determined using the formula given below.

$$\% Loading efficiency = \frac{Total Afatinib used - Afatinib in supernatant}{Total Afatinib used} \times 100$$

### (17) Hemocompatibility Studies

### Hemocompatibility

Human blood from donors was collected and handled by following methods. Informed consent was obtained from the study participants before drawing blood for the hemocompatibility studies. After collection, human blood was incubated with CAR-T-MNPs at different concentrations (0, 250, 500, 750, and 1000 μg/ml), 0.9% saline (negative control), or distilled water (positive control) for 2 hours. Following centrifugation at 1000 g, absorbance readings were taken at 545 nm. The percentage of hemolysis was calculated using the equation below:

$$\% Hemolysis = \frac{( Sample OD - Negative Control OD )}{( Positive Control OD - Negative Control OD )} \times 100$$

To study blood clotting kinetics, CAR-T-MNPs of varying concentrations (0, 250, 500, 750, and 1000 μg/ml) were added to blood that was activated with CaCl2 (n=9). At pre-determined time points (10, 20, 30, and 60 minutes), the red blood cells (RBCs) not involved in clot formation were lysed. Absorbance readings of supernatant were taken at 540 nm. The absorbance readings were inversely proportional to clot formation.

### Cytocompatibility

AT1 cells were seeded (5×10³ cells/well) in 96-well plates and incubated overnight at 37° C. CAR-T-MNPs were added at concentrations ranging from 0-1000 μg/mL to the seeded cells. After incubation for 48 hours, cells were incubated with MTT reagent (Promega) for one hour followed by measurement of the absorbance at 490 nm using an UV-Vis spectrophotometer (Tecan). Cell viabilities were calculated via normalizing to the control group (cells exposed to complete media only were considered having a 100% viability).

### (18) Binding Kinetics of Fabricated Anti HER-2 PLGA NPs Via Competitive ELISA Assays

To measure whether fabricated NPs were able to engage with HER-2 protein, competitive ELISA assays were performed with anti HER-2 conjugated PLGA NPs. Microtiter ELISA plate wells were coated with 100 μl of the HER-2 antigen solution at a concentration of 0.1 μg/ml in coating buffer. Plates were incubated overnight at 4° C., washed with 300 μl wash buffer three times, and blocked with 300 μl blocking buffer at 37° C. for 1.5 hours. Plates were washed 3 times and incubated with different concentrations of anti HER-2 antibody conjugated PLGA NPs in the presence of biotinylated anti HER-2 antibodies (0.05 μl/ml) for one hour at 37° C. After repeating the washing steps, 100 μl of HRP-conjugated streptavidin (0.1 μg/ml in dilution buffer) was added to each well and incubated for 1 hour at 37° C. After the washing steps, 100 μl TMB substrate solution was added to each well and incubated at room temperature for 30 minutes, and then the absorbance values were measured on 450 nm wavelength (Tecan). Data was normalized against bare PLGA NPs and analyzed for experimental and control groups.

### (19) In Vitro Cellular Uptake Studies

For fluorescent uptake microscopy imaging (EVOS Floid, Invitrogen) studies, A549 were seeded on a glass bottomed six well tissue culture plate (Corning). Jurkat-T-MNPs, anti HER-2-PLGA NPs or CAR-T-MNPs were added and incubated with cells for 30 minutes at 37° C. Cells were then washed using PBS and were fixed using 5% formaldehyde for 20 minutes. The cell nuclei were stained using NucBlue (Thermo), and cells were imaged using the inverted fluorescent microscopy. For the spectrophotometric analysis of uptake, Coumarin-6 (C-6; fluorescent drug) loaded Jurkat-T-MNPs, anti HER-2 PLGA NPs and CAR-T-MNPs at different NP concentrations (100 μg/mL, 250 μg/mL, 500 μg/mL, 750 μg/mL) were exposed to cells in 96 well plates for 30 minutes and subsequently washed with 1×PBS and lysed with 250 μl/well of 1% Triton® X-100 (approximately 30 min incubation). Cell extracts were then analyzed for protein content using the Pierce BCA protein assay kit (Thermo Scientific, Rockford, IL) and C-6 fluorescent intensity (emitted from particles uptaken inside the cell) using a UV-vis Spectrophotometer (458/540 em/ex). Total protein concentration in each lysate was calculated using a BSA standard curve. The uptake of the nanoparticles was calculated by normalizing the particle concentration (determined from fluorescence intensity in a lysate) in each sample with total cell protein, which correlated to the number of cells in the sample.

### (20) In Vitro Cytotoxicity Assays

A549, MDA-MB-231 and SKOV-3 cells were seeded (5×10³ cells/well) in 96-well plates and incubated overnight. Free Afatinib, Afatinib loaded PLGA NPs, anti HER-2 PLGA NPs, Jurkat-T-MNPs, and CAR-T-MNPs were added to the seeded cells (n=4). Alternatively, free cisplatin, cisplatin loaded PLGA NPs, Jurkat-T-MNPs, and CAR-T-MNPs were added to the seeded cells (n=4) at a cisplatin concentration of 7 μM (A549) & 3.5 μM (SKOV-3). After incubation for 48 hours, the absorbance at 490 nm was measured using UV-VIS Spectrophotometer (Tecan) after incubating with MTT reagent (Promega) for one hour. Cell viabilities were calculated via normalizing to the control group (cells exposed to complete media, 100% viability).

### (21) Animal Model

Nude mice (NU/J) aged 5-6 weeks were purchased from Jacksons Laboratory. Briefly, 2×10⁶ A549 cells in 200 μL of Matrigel (Corning, NY) were injected subcutaneously in athymic nude mice. Tumor volumes were checked at regular intervals of 3 days and treatment started when the volume reached ˜300 mm3. Tumor volumes were measured using a standard Vernier Calipers. All procedures involving experimental animals were performed in accordance with the protocols approved by the University of Texas at Arlington Institutional Animal Care and Use Committee.

### (22) In Vitro Live-Dead Assays

A549 cells were seeded at a density of 5×10³ cells/well in 96-well plates and incubated overnight. Free Afatinib, Afatinib loaded PLGA NPs, anti HER-2 conjugated PLGA NPs, Jurkat-T-MNPs, and CAR-T-MNPs were added to the seeded cells (n=4). After incubation for 48 hours, cells were stained with enzo live-dead staining kits following the company's instructions. Cells were incubated with permeable Live-Dye™ and the cell non-permeable PI (red) dyes in staining buffer for 15 minutes at 37° C. Following incubation with the staining solution, cells were visualized using the inverted fluorescent microscope, and images were recorded.

### (23) In Vivo Biodistribution of CAR-T-MNPs in Tumor Implanted Mice

The biodistribution study was performed to evaluate targeting efficiency of intravenously injected CAR-T-MNPs towards subcutaneous tumors implanted in nude mice. ICG-loaded CAR-T-MNPs were prepared in the same formulation procedure aforementioned. Saline, ICG-loaded PLGA NPs, Jurkat-T-MNPs and CAR-T-MNPs were intravenously given to mice at 5 mg of dye (ICG) loaded particles. At 1, 4, 8, and 16 hours, images of animals for all groups were monitored and recorded by the Kodak FX/Pro imaging system. After the last time point (16 hours) of imaging, mice were autopsied, and tumors and major organs were carefully collected and weighed. The mice were euthanized at the end of the treatment and the tumor tissue was collected and fixed with 4% Paraformaldehyde. The fluorescence signals of tumors and each organ were recoded using the Kodak FX/Pro imaging system for quantification. Furthermore, weighted tumors and organs were homogenized for 2 minutes using a tabletop Precellys homogenizer (Bertin Instruments). Homogenates were centrifuged at 4000 rpm for 10 minutes, and fluorescent measurements of supernatants were taken at λₑₓ 786 nm and λₑₘ 825 nm using a spectrophotometer (Tecan). Effective nanoparticle accumulation in all organs and tumors (μg NP per mg of organ) were determined using a standard curve of ICG PLGA NPs and normalized against the saline group.

### (24) In Vivo Therapeutic Efficiency of CAR-T-MNPs

Xenograft tumors as described above were used to evaluate the therapeutic efficiency of CAR-T-MNPs. After the tumor reached ˜300 mm3, the mice were randomly distributed into 4 groups (n=3) for different treatment groups. Prior to treatment, all mice were ear-tagged, the initial tumor volumes were measured by a caliper, and the body weights were recorded. The treatment groups received a single tail-vein IV administration of either of 200 μL of saline (control), free drug, Jurkat-T-MNPs or CAR-T-MNPs. Tumor volume was measured every 2-3 days via a caliper. Tumor volumes were calculated by measuring the tumor perpendicular diameters with a caliper and calculated using the formula V=½×a×b2, where a and b are the longest diameter and its longest perpendicular diameter, respectively. Tumor growth was monitored until the longest diameter reached 20 mm or the animals exhibited unusual behavior, at which point they were euthanized by carbon dioxide.

The mice were euthanized after the treatment end point and the tumor tissue was collected and fixed with Formalin. The tissue was embedded in paraffin wax and sectioned into 5 um sections, using Shandon Finnesse (Thermofisher) followed by further histological examinations using the hematoxylin and eosin (H&E) staining.

### (25) Statistical Analysis

GraphPad Prism 9 (GraphPad Software Inc., San Diego) was used to perform statistical analysis. Two-way ANOVA with Tukey's multiple comparisons test was done for all the analyses. Triplicate samples were used for all the studies. (*P<0.05, **P<0.01, ***P<0.001, ****P<0.0001).

### Example 2: Bi-Specific CAR

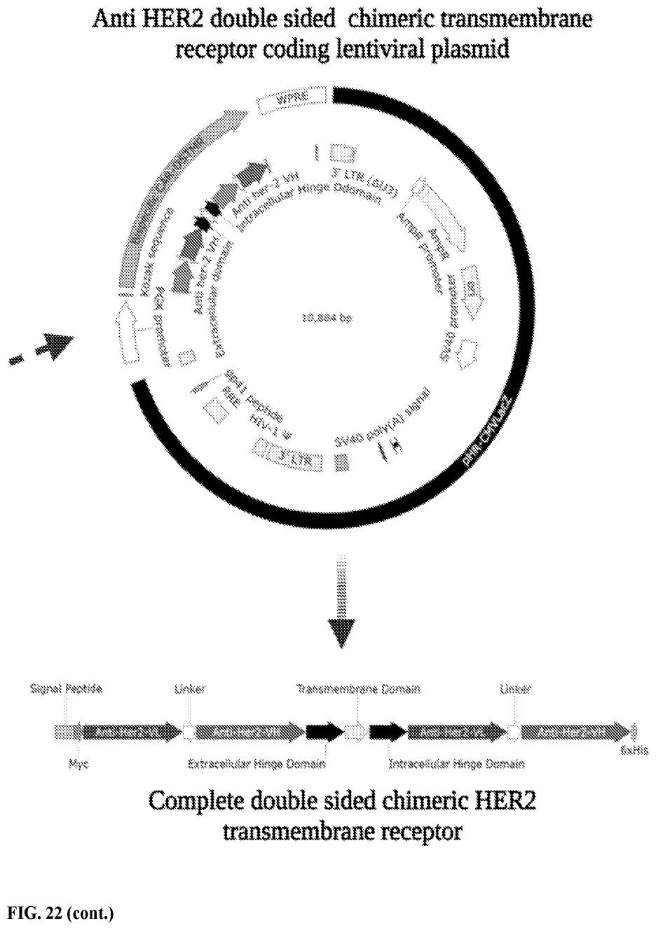

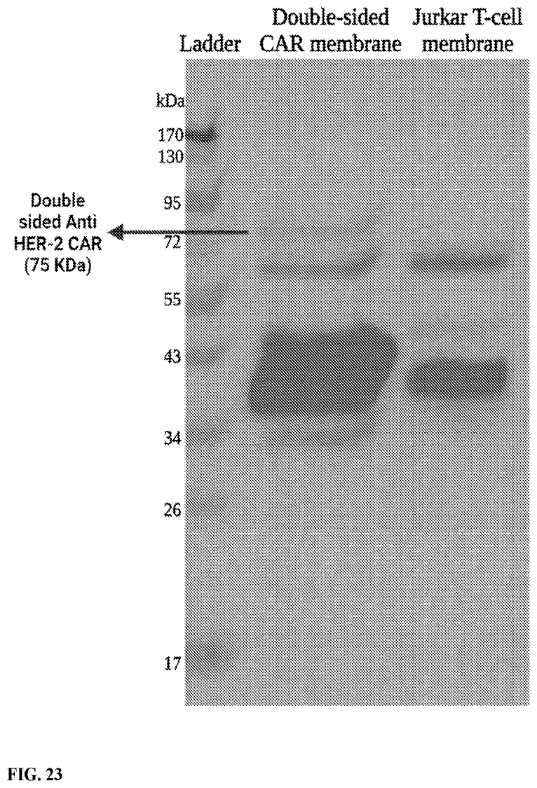

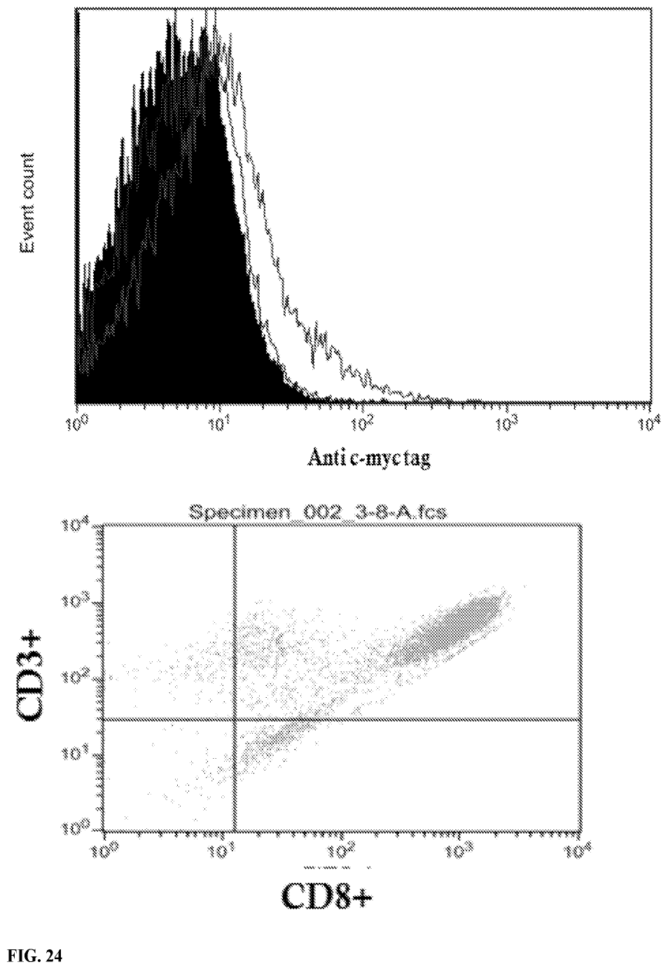

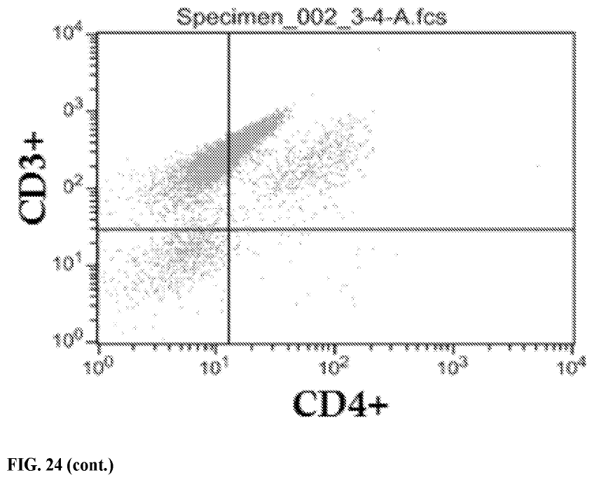

Although cell membrane coated nanocarriers can deliver drugs at the desired location, there are limitations and challenges associated with the approach. The cell membrane is a unique structure and comprised of various molecules (lipids, proteins, receptors, ligands, sugar moieties etc.), some of which are required for targeting, evading immune response while the other abundant proteins might have still unknown interactions in the host environment. Isolation of cell membranes is still limited to laboratory and quality controls such as maintaining the functional and structural aspects of cell membranes during membrane isolation techniques need to be investigated for clinical translation. For example, during the extrusion process, inside-out or outside-in oriented membrane vesicles are possibly coating the PLGA nanoparticles, and this reduces the presence of targeting moiety on the surface of the particles, leading to lower targeting efficacy. One solution to this phenomenon is to engineer cells CAR molecules to have both intra- and extracellular facing ScFv's (bidirectional CARs). This new strategy (FIG. 22) is a new way to further improve the existing design. By this way, isolated membranes have targeting ScFv's on both sides of their lipid bilayer and when extruded along with NPs, the outer layer of the membrane coated NPs always have targeting ScFv's. With this method, targeting molecule number per carrier can be increased on the particle surface via integrating these “bidirectional CARs” (BiCARs). Thus, BiCARs engineered membranes can drastically improve the outcomes of proposed research. For this purpose, studies have been done and the assessment of double-sided CAR molecules on the cells using western blot (FIG. 23). Further characterization of BiCAR including extrusion with NPs, targeting efficacy, and biodistribution profiles are under investigation.

To fulfill this need we have developed and characterized CAR engineered cell membrane coated synthetic nano particles. Our CAR-T-MNPs displayed a high drug encapsulation efficiency of 61% and can release the drug in a sustained fashion over a period of 21 days. The drug release profile was found to be dependent on the existence of a membrane on the particles in which the membrane coated nanoparticles provided a slower release due to the barrier effect. Fabricated CAR-T-MNPs were shown to be suitable for IV administration in terms of their size (˜200 nm), cell compatibility, stability and hemocompatibility. Moreover, cellular uptake studies showed selective uptake by HER2 positive lung cancer cells when compared to jurkat cell membrane coated PLGA and bare PLGA NPs. These findings also correlated with enhanced therapeutic efficiencies of CAR-T-MNPs. Therefore, CAR engineered membrane coated NPs system are a promising cell mimicking drug carrier that can improve the therapeutic outcomes of non-small lung cancer treatments. On the other hand, high systemic side effects have always been the major concern of chemotherapeutic drugs. The developed CAR-T technology proved to be an effective and safe alternative. As shown in our pre-clinical results, the CAR-T-MNPs have a very high targeting and retention towards tumor tissue with less non-specific organ targeting. The CAR-T-MNP technology also reduced tumor growth with just a single dose treatment.

### G. REFERENCES

Androulla, M. N. and P. C. Lefkothea, CAR T-cell therapy: a new era in cancer immunotherapy. Current Pharmaceutical Biotechnology, 2018. 19(1): p. 5-18.
Angsantikul, P., et al., Cell membrane-coated nanoparticles as an emerging antibacterial vaccine platform. Vaccines, 2015. 3(4): p. 814-828.
Athey, V. L.; Suckling, R. J.; Tod, A. M.; Walters, S. J.; Rogers, T. K., Early diagnosis of lung cancer: evaluation of a community-based social marketing intervention. Thorax 2012, 67 (5), 412-417.
Babu, A., et al., Nanoparticle-based drug delivery for therapy of lung cancer: progress and challenges. Journal of Nanomaterials, 2013. 2013: p. 14.
Baek, S.-K., et al., Photothermal treatment of glioma; an in vitro study of macrophage-mediated delivery of gold nanoshells. Journal of Neuro-Oncology, 2011. 104(2): p. 439-448.
Baeumler, T. A., A. A. Ahmed, and T. A. Fulga, Engineering synthetic signaling pathways with programmable dCas9-based chimeric receptors. Cell Reports, 2017. 20(11): p. 2639-2653.
Baggiolini, M., Chemokines and leukocyte traffic. Nature, 1998. 392(6676): p. 565.
Bahmani, B., D. Bacon, and B. Anvari, Erythrocyte-derived photo-theranostic agents: hybrid nano-vesicles containing indocyanine green for near infrared imaging and therapeutic applications. Scientific Reports, 2013. 3: p. 2180.
Barenholz, Y., Sphingomyelin and cholesterol: from membrane biophysics and rafts to potential medical applications, in Membrane Dynamics and Domains. 2004, Springer. P. 167-215.
Bartlett, D. W., et al., Impact of tumor-specific targeting on the biodistribution and efficacy of siRNA nanoparticles measured by multimodality in vivo imaging. Proceedings of the National Academy of Sciences, 2007. 104(39): p. 15549-15554.
Basotra, M.; Singh, S. K.; Gulati, M., Development and Validation of a Simple and Sensitive Spectrometric Method for Estimation of Cisplatin Hydrochloride in Tablet Dosage Forms: Application to Dissolution Studies. ISRN Analytical Chemistry 2013, 2013, 936254.
Beatty, G. L. and W. L. Gladney, Immune escape mechanisms as a guide for cancer immunotherapy. Clinical Cancer Research, 2015. 21(4): p. 687-692.
Besse, B.; Le Chevalier, T., Developments in the treatment of early NSCLC: when to use chemotherapy. Ann Oncol 2012, 23 Suppl 10, x52-9.
Biswas, S., et al., Liposomes loaded with paclitaxel and modified with novel triphenylphosphonium-PEG-PE conjugate possess low toxicity, target mitochondria and demonstrate enhanced antitumor effects in vitro and in vivo. Journal of Controlled Release, 2012. 159(3): p. 393-402.
Brannon-Peppas, L. and J. O. Blanchette, Nanoparticle and targeted systems for cancer therapy. Advanced drug delivery reviews, 2012. 64: p. 206-212.
Bu, L. L., et al., Cancer stem cell-platelet hybrid membrane-coated magnetic nanoparticles for enhanced photothermal therapy of head and neck squamous cell carcinoma. Advanced Functional Materials, 2019. 29(10): p. 1807733.
Bunn, P. A., et al., Expression of Her-2/neu in human lung cancer cell lines by immunohistochemistry and fluorescence in situ hybridization and its relationship to in vitro cytotoxicity by trastuzumab and chemotherapeutic agents. Clinical Cancer Research, 2001. 7(10): p. 3239-3250.
Cao, H., et al., Liposomes coated with isolated macrophage membrane can target lung metastasis of breast cancer. ACS Nano, 2016. 10(8): p. 7738-7748.
Cao, X., et al., Neutrophil-mimicking therapeutic nanoparticles for targeted chemotherapy of pancreatic carcinoma. Acta Pharmaceutica Sinica B, 2019. 9(3): p. 575-589.
Cho, J. H., Immunotherapy for non-small-cell lung cancer: current status and future obstacles. Immune Network, 2017. 17(6): p. 378-391.
Colombo, G., et al., Structure-activity relationships of linear and cyclic peptides containing the NGR tumor-homing motif. Journal of Biological Chemistry, 2002. 277(49): p. 47891-47897.
Conde, J., G. Doria, and P. Baptista, Noble metal nanoparticles applications in cancer. Journal of Drug Delivery, 2012. 2012.
Corbo, C., et al., The impact of nanoparticle protein corona on cytotoxicity, immunotoxicity and target drug delivery. Nanomedicine, 2016. 11(1): p. 81-100.
Crittenden, M., et al., Pharmacologically regulated production of targeted retrovirus from T cells for systemic antitumor gene therapy. Cancer Research, 2003. 63(12): p. 3173-3180.
Danesh, A., et al., Exosomes from red blood cell units bind to monocytes and induce proinflammatory cytokines, boosting T-cell responses in vitro. Blood, 2014. 123(5): p. 687-696.
Dehaini, D., et al., Erythrocyte-platelet hybrid membrane coating for enhanced nanoparticle functionalization. Advanced Materials, 2017. 29(16): p. 1606209.
Detterbeck, F. C., D. J. Boffa, and L. T. Tanoue, The new lung cancer staging system. Chest, 2009. 136(1): p. 260-271.
Domínguez-Ríos, R.; Sánchez-Ramírez, D. R.; Ruiz-Saray, K.; Oceguera-Basurto, P. E.; Almada, M.; Juárez, J.; Zepeda-Moreno, A.; del Toro-Arreola, A.; Topete, A.; Daneri-Navarro, A., Cisplatin-loaded PLGA nanoparticles for HER2 targeted ovarian cancer therapy. Colloids and Surfaces B: Biointerfaces 2019, 178, 199-207.
Elegheert, J.; Behiels, E.; Bishop, B.; Scott, S.; Woolley, R. E.; Griffiths, S. C.; Byrne, E. F. X.; Chang, V. T.; Stuart, D. I.; Jones, E. Y.; Siebold, C.; Aricescu, A. R., Lentiviral transduction of mammalian cells for fast, scalable and high-level production of soluble and membrane proteins. Nature Protocols 2018, 13 (12), 2991-3017.
Elias, D. R., et al., Effect of ligand density, receptor density, and nanoparticle size on cell targeting. Nanomedicine: Nanotechnology, Biology and Medicine, 2013. 9(2): p. 194-201.
Escamilla-Rivera, V., et al., Protein corona acts as a protective shield against Fe3O4-PEG inflammation and ROS-induced toxicity in human macrophages. Toxicology Letters, 2016. 240(1): p. 172-184.
Fang, F., W. Xiao, and Z. Tian. NK cell-based immunotherapy for cancer. In Seminars in immunology. 2017. Elsevier.
Fang, R. H., et al., Cancer cell membrane-coated nanoparticles for anticancer vaccination and drug delivery. Nano Letters, 2014. 14(4): p. 2181-2188.
Fang, R. H., et al., Cell membrane coating nanotechnology. Advanced Materials, 2018. 30(23): p. 1706759.
Fantappie, L., et al., Antibody-mediated immunity induced by engineered Escherichia coli OMVs carrying heterologous antigens in their lumen. Journal of Extracellular Vesicles, 2014. 3(1): p. 24015.
Felgner, S., et al., Engineered Salmonella enterica serovar Typhimurium overcomes limitations of anti-bacterial immunity in bacteria-mediated tumor therapy. Oncoimmunology, 2018. 7(2): p. e1382791.
Feliu, N., et al., In vivo degeneration and the fate of inorganic nanoparticles. Chemical Society Reviews, 2016. 45(9): p. 2440-2457.
Fernández-Ponce, C.; Durán-Ruiz, M. C.; Narbona-Sánchez, I.; Muñoz-Miranda, J. P.; Arbulo-Echevarria, M. M.; Serna-Sanz, A.; Baumann, C.; Litrán, R.; Aguado, E.; Bloch, W.; García-Cozar, F., Ultrastructural Localization and Molecular Associations of HCV Capsid Protein in Jurkat T Cells. Frontiers in Microbiology 2018, 8.
Fontana, F., et al., Multistaged nanovaccines based on porous silicon@ acetalated dextran@ cancer cell membrane for cancer immunotherapy. Advanced Materials, 2017. 29(7): p. 1603239.
Fukuoka, M.; Yano, S.; Giaccone, G.; Tamura, T.; Nakagawa, K.; Douillard, J. Y.; Nishiwaki, Y.; Vansteenkiste, J.; Kudoh, S.; Rischin, D.; Eek, R.; Horai, T.; Noda, K.; Takata, I.; Smit, E.; Averbuch, S.; Macleod, A.; Feyereislova, A.; Dong, R. P.; Baselga, J., Multi-institutional randomized phase II trial of gefitinib for previously treated patients with advanced non-small-cell lung cancer (The IDEAL 1 Trial) [corrected]. J Clin Oncol 2003, 21 (12), 2237-46.
Gajewski, T. F., H. Schreiber, and Y.-X. Fu, Innate and adaptive immune cells in the tumor microenvironment. Nature Immunology, 2013. 14(10): p. 1014.
Gao, C., et al., Stem cell membrane-coated nanogels for highly efficient in vivo tumor targeted drug delivery. Small, 2016. 12(30): p. 4056-4062.
Gao, M., et al., Erythrocyte-Membrane-Enveloped Perfluorocarbon as Nanoscale Artificial Red Blood Cells to Relieve Tumor Hypoxia and Enhance Cancer Radiotherapy. Advanced Materials, 2017. 29(35): p. 1701429.
Gao, W., et al., Modulating antibacterial immunity via bacterial membrane-coated nanoparticles. Nano Letters, 2015. 15(2): p. 1403-1409.
Gao, W., et al., Surface functionalization of gold nanoparticles with red blood cell membranes. Advanced Materials, 2013. 25(26): p. 3549-3553.
Ghitescu, L. and M. Bendayan, Immunolabeling efficiency of protein A-gold complexes. Journal of Histochemistry & Cytochemistry, 1990. 38(11): p. 1523-1530.
Ghoneim, H. E., et al., Cell-intrinsic barriers of T cell-based immunotherapy. Trends in Molecular Medicine, 2016. 22(12): p. 1000-1011.
Gong, P.; Wang, Y.; Zhang, P.; Yang, Z.; Deng, W.; Sun, Z.; Yang, M.; Li, X.; Ma, G.; Deng, G.; Dong, S.; Cai, L.; Jiang, W., Immunocyte Membrane-Coated Nanoparticles for Cancer Immunotherapy. Cancers 2021, 13 (1).
Gujrati, V., et al., Bioengineered bacterial outer membrane vesicles as cell-specific drug-delivery vehicles for cancer therapy. ACS Nano, 2014. 8(2): p. 1525-1537.
Guo, J., et al., Aptamer-functionalized PEG-PLGA nanoparticles for enhanced anti-glioma drug delivery. Biomaterials, 2011. 32(31): p. 8010-8020.
Hillman, G. G.; Lonardo, F.; Hoogstra, D. J.; Rakowski, J.; Yunker, C. K.; Joiner, M. C.; Dyson, G.; Gadgeel, S.; Singh-Gupta, V., Axitinib Improves Radiotherapy in Murine Xenograft Lung Tumors. Translational Oncology 2014, 7 (3), 400-409.
Hirsh, V., Next-generation covalent irreversible kinase inhibitors in NSCLC: focus on afatinib. BioDrugs, 2015. 29(3): p. 167-183.
Hong, M., et al., Novel anti-tumor strategy: PEG-hydroxycamptothecin conjugate loaded transferrin-PEG-nanoparticles. Journal of Controlled Release, 2010. 141(1): p. 22-29.
Houot, R., et al., T-cell-based immunotherapy: adoptive cell transfer and checkpoint inhibition. Cancer Immunology Research, 2015. 3(10): p. 1115-1122.
Hu, C.-M. J., et al., A biomimetic nanosponge that absorbs pore-forming toxins. Nature Nanotechnology, 2013. 8(5): p. 336.
Hu, C.-M. J., et al., Erythrocyte membrane-camouflaged polymeric nanoparticles as a biomimetic delivery platform. Proceedings of the National Academy of Sciences, 2011. 108(27): p. 10980-10985.
Huang, Y., X. Gao, and J. Chen, Leukocyte-derived biomimetic nanoparticulate drug delivery systems for cancer therapy. Acta Pharmaceutica Sinica B, 2018. 8(1): p. 4-13.
Hühn, J., et al., Dissociation coefficients of protein adsorption to nanoparticles as quantitative metrics for description of the protein corona: A comparison of experimental techniques and methodological relevance. The International Journal of Biochemistry & Cell Biology, 2016. 75: p. 148-161.
Immordino, M. L., F. Dosio, and L. Cattel, Stealth liposomes: review of the basic science, rationale, and clinical applications, existing and potential. International Journal of Nanomedicine, 2006. 1(3): p. 297.
Irvine, D. J., M. A. Swartz, and G. L. Szeto, Engineering synthetic vaccines using cues from natural immunity. Nature Materials, 2013. 12(11): p. 978.
Jin, J., et al., Human cancer cell membrane coated biomimetic nanoparticles reduce fibroblast-mediated invasion and metastasis, and induce T cells. ACS Applied Materials & Interfaces, 2019.
Juweid, M., et al., Micropharmacology of monoclonal antibodies in solid tumors: direct experimental evidence for a binding site barrier. Cancer Research, 1992. 52(19): p. 5144-5153.
Kang, T., et al., Nanoparticles coated with neutrophil membranes can effectively treat cancer metastasis. ACS Nano, 2017. 11(2): p. 1397-1411.
Karmakar, S., Cell Based Immunotherapy: As a Promising Futuristic Solution for Effective Cancer Therapy. Single Cell Biol, 2014. 4(105): p. 2.
Kim, O. Y., et al., Bacterial outer membrane vesicles suppress tumor by interferon-γ-mediated antitumor response. Nature Communications, 2017. 8(1): p. 626.
Kona, S.; Dong, J.-F.; Liu, Y.; Tan, J.; Nguyen, K. T., Biodegradable nanoparticles mimicking platelet binding as a targeted and controlled drug delivery system. International Journal of Pharmaceutics 2012, 423 (2), 516-524.
Krishnamurthy, S., et al., Monocyte cell membrane-derived nanoghosts for targeted cancer therapy. Nanoscale, 2016. 8(13): p. 6981-6985.
Kroll, A. V., et al., Nanoparticulate delivery of cancer cell membrane elicits multiantigenic antitumor immunity. Advanced Materials, 2017. 29(47): p. 1703969.
Kroll, A. V., R. H. Fang, and L. Zhang, Biointerfacing and applications of cell membrane-coated nanoparticles. Bioconjugate Chemistry, 2016. 28(1): p. 23-32.
Kuehn, M. J. and N. C. Kesty, Bacterial outer membrane vesicles and the host-pathogen interaction. Genes & Development, 2005. 19(22): p. 2645-2655.
Lambour, J., et al., Converting monoclonal antibody-based immunotherapies from passive to active: bringing immune complexes into play. Emerging Microbes & Infections, 2016. 5(8): p. e92.
Langer, R. and D. A. Tirrell, Designing materials for biology and medicine. Nature, 2004. 428(6982): p. 487.
Lee, E. Y., et al., Global proteomic profiling of native outer membrane vesicles derived from Escherichia coli. Proteomics, 2007. 7(17): p. 3143-3153.
Ley, K., Molecular mechanisms of leukocyte recruitment in the inflammatory process. Cardiovascular Research, 1996. 32(4): p. 733-742.
Li, R., et al., Cell membrane-based nanoparticles: a new biomimetic platform for tumor diagnosis and treatment. Acta Pharmaceutica Sinica B, 2017.
Li, R., et al., Cell membrane-based nanoparticles: a new biomimetic platform for tumor diagnosis and treatment. Acta Pharmaceutica Sinica B, 2018. 8(1): p. 14-22.
Lindner, L. H., et al., Novel temperature-sensitive liposomes with prolonged circulation time. Clinical Cancer Research, 2004. 10(6): p. 2168-2178.
Luk, B. T., et al., Safe and immunocompatible nanocarriers cloaked in RBC membranes for drug delivery to treat solid tumors. Theranostics, 2016. 6(7): p. 1004.
Lundqvist, M., et al., Nanoparticle size and surface properties determine the protein corona with possible implications for biological impacts. Proceedings of the National Academy of Sciences, 2008. 105(38): p. 14265-14270.
Ma, W.; Zhu, D.; Li, J.; Chen, X.; Xie, W.; Jiang, X.; Wu, L.; Wang, G.; Xiao, Y.; Liu, Z.; Wang, F.; Li, A.; Shao, D.; Dong, W.; Liu, W.; Yuan, Y., Coating biomimetic nanoparticles with chimeric antigen receptor T cell-membrane provides high specificity for hepatocellular carcinoma photothermal therapy treatment. Theranostics 2020, 10 (3), 1281-1295.
Ma, Y. Q., J. Qin, and E. Plow, Platelet integrin αIIbβ3: activation mechanisms. Journal of Thrombosis and Haemostasis, 2007. 5(7): p. 1345-1352.
Maciejczyk, A., I. Skrzypczyńska, and M. Janiszewska, Lung cancer. Radiotherapy in lung cancer: Actual methods and future trends. Reports of Practical Oncology & Radiotherapy, 2014. 19(6): p. 353-360.
Mahmoudi, M., Protein corona: The golden gate to clinical applications of nanoparticles. International Journal of Biochemistry and Cell Biology, 2016(75): p. 141-142.
Manchester, M. and P. Singh, Virus-based nanoparticles (VNPs): platform technologies for diagnostic imaging. Advanced Drug Delivery Reviews, 2006. 58(14): p. 1505-1522.
Mengesha, A., et al., Potential and limitations of bacterial-mediated cancer therapy. Front Biosci, 2007. 12: p. 3880-3891.
Merkel, T. J., et al., Using mechanobiological mimicry of red blood cells to extend circulation times of hydrogel microparticles. Proceedings of the National Academy of Sciences, 2011. 108(2): p. 586-591.
Mitchell, M. J.; Billingsley, M. M.; Haley, R. M.; Wechsler, M. E.; Peppas, N. A.; Langer, R., Engineering precision nanoparticles for drug delivery. Nature Reviews Drug Discovery 2021, 20 (2), 101-124.
Mitchell, M. J. and M. R. King, Leukocytes as carriers for targeted cancer drug delivery. Expert Opinion on Drug Delivery, 2015. 12(3): p. 375-392.
Mitchell, M. J., et al., TRAIL-coated leukocytes that kill cancer cells in the circulation. Proceedings of the National Academy of Sciences, 2014. 111(3): p. 930-935.
Moghimi, S. M. and J. Szebeni, Stealth liposomes and long circulating nanoparticles: critical issues in pharmacokinetics, opsonization and protein-binding properties. Progress in Lipid Research, 2003. 42(6): p. 463-478.
Mohr, A. and R. Zwacka, The future of mesenchymal stem cell-based therapeutic approaches for cancer—From cells to ghosts. Cancer Letters, 2017.
Molina, J. R., et al. Non-small cell lung cancer: epidemiology, risk factors, treatment, and survivorship. In Mayo Clinic Proceedings. 2008. Elsevier.
Monopoli, M. P., et al., Physical-chemical aspects of protein corona: relevance to in vitro and in vivo biological impacts of nanoparticles. Journal of the American Chemical Society, 2011. 133(8): p. 2525-2534.
Morera, D. and S. A. Mackenzie, Is there a direct role for erythrocytes in the immune response? Veterinary Research, 2011. 42(1): p. 89.
Munich, S., et al., Dendritic cell exosomes directly kill tumor cells and activate natural killer cells via TNF superfamily ligands. Oncoimmunology, 2012. 1(7): p. 1074-1083.
Muralinath, M., et al., Immunization with Salmonella enterica serovar Typhimurium-derived outer membrane vesicles delivering the pneumococcal protein PspA confers protection against challenge with Streptococcus pneumoniae. Infection and Immunity, 2011. 79(2): p. 887-894.
Muro, S., Challenges in design and characterization of ligand-targeted drug delivery systems. Journal of Controlled Release, 2012. 164(2): p. 125-137.
Nukolova, N. V., et al., Folate-decorated nanogels for targeted therapy of ovarian cancer. Biomaterials, 2011. 32(23): p. 5417-5426.
Ohguri, T.; Imada, H.; Yahara, K.; Moon, S. D.; Yamaguchi, S.; Yatera, K.; Mukae, H.; Hanagiri, T.; Tanaka, F.; Korogi, Y., Re-irradiation plus regional hyperthermia for recurrent non-small cell lung cancer: a potential modality for inducing long-term survival in selected patients. Lung cancer (Amsterdam, Netherlands) 2012, 77 (1), 140-5.
Ohguri, T.; Imada, H.; Yahara, K.; Morioka, T.; Nakano, K.; Terashima, H.; Korogi, Y., Radiotherapy with 8-MHz radiofrequency-capacitive regional hyperthermia for stage III non-small-cell lung cancer: the radiofrequency-output power correlates with the intraesophageal temperature and clinical outcomes. International journal of radiation oncology, biology, physics 2009, 73 (1), 128-35.
Orbach, A., et al., Biophysical and biochemical markers of red blood cell fragility. Transfusion Medicine and Hemotherapy, 2017. 44(3): p. 183-187.
Panés, J., M. Perry, and D. N. Granger, Leukocyte-endothelial cell adhesion: avenues for therapeutic intervention. British Journal of Pharmacology, 1999. 126(3): p. 537-550.
Pardoll, D. M., The blockade of immune checkpoints in cancer immunotherapy. Nature Reviews Cancer, 2012. 12(4): p. 252.
Parodi, A., et al., Bio-inspired engineering of cell- and virus-like nanoparticles for drug delivery. Biomaterials, 2017. 147: p. 155-168.
Parodi, A., et al., Synthetic nanoparticles functionalized with biomimetic leukocyte membranes possess cell-like functions. Nature Nanotechnology, 2013. 8(1): p. 61.
Pasto, A., et al., Cell membrane protein functionalization of nanoparticles as a new tumor-targeting strategy. Clinical and translational medicine, 2019. 8(1): p. 8.
Paugh, B. S.; Baranyi, L.; Roy, A.; He, H.-J.; Harris, L.; Cole, K. D.; Artlip, M.; Raimund, C.; Langan, P. S.; Jana, S.; Orentas, R. J.; Lin-Gibson, S.; Krueger, W.; Dropulić, B., Reference standards for accurate validation and optimization of assays that determine integrated lentiviral vector copy number in transduced cells. Scientific Reports 2021, 11 (1), 389.
Paulitschke, M., et al., Perturbation of red blood cell membrane rigidity by extracellular ligands. Blood, 1995. 86(1): p. 342-348.
Peer, D., et al., Nanocarriers as an emerging platform for cancer therapy. Nature Nanotechnology, 2007. 2(12): p. 751.
Pérez-Herrero, E. and A. Fernández-Medarde, Advanced targeted therapies in cancer: drug nanocarriers, the future of chemotherapy. European Journal of Pharmaceutics and Biopharmaceutics, 2015. 93: p. 52-79.
Pho-iam, T.; Punnakitikashem, P.; Somboonyosdech, C.; Sripinitchai, S.; Masaratana, P.; Sirivatanauksorn, V.; Sirivatanauksorn, Y.; Wongwan, C.; Nguyen, K. T.; Srisawat, C., PLGA nanoparticles containing a-fetoprotein siRNA induce apoptosis and enhance the cytotoxic effects of doxorubicin in human liver cancer cell line. Biochemical and Biophysical Research Communications 2021, 553, 191-197.
Piao, J.-G., et al., Erythrocyte membrane is an alternative coating to polyethylene glycol for prolonging the circulation lifetime of gold nanocages for photothermal therapy. ACS Nano, 2014. 8(10): p. 10414-10425.
Pirollo, K. F. and E. H. Chang, Does a targeting ligand influence nanoparticle tumor localization or uptake? Trends in Biotechnology, 2008. 26(10): p. 552-558.
Poetsch, A. and D. Wolters, Bacterial membrane proteomics. Proteomics, 2008. 8(19): p. 4100-4122.
Qiu, L., et al., A cell-targeted, size-photocontrollable, nuclear-uptake nanodrug delivery system for drug-resistant cancer therapy. Nano Letters, 2014. 15(1): p. 457-463.
Raez, L. E., S. Fein, and E. R. Podack, Lung cancer immunotherapy. Clinical Medicine & Research, 2005. 3(4): p. 221-228.
Rami-Porta, R., et al., Complete resection in lung cancer surgery: proposed definition. Lung Cancer, 2005. 49(1): p. 25-33.
Ramzy, L., et al., Cancer nanotheranostics: a review of the role of conjugated ligands for overexpressed receptors. European Journal of Pharmaceutical Sciences, 2017. 104: p. 273-292.
Ranganathan, R. and M. C. Foster, The Limitations and Promise of Immunotherapy With Chimeric Antigen-Modified T Cells. 2016, UBM MEDICA 535 CONNECTICUT AVE, STE 300, NORWALK, CT 06854 USA.
Roybal, K. T., et al., Engineering T cells with customized therapeutic response programs using synthetic notch receptors. Cell, 2016. 167(2): p. 419-432. E16.
Sabado, R. L., S. Balan, and N. Bhardwaj, Dendritic cell-based immunotherapy. Cell Research, 2017. 27(1): p. 74.
Sadhukha, T., T. D. O'brien, and S. Prabha, Nano-engineered mesenchymal stem cells as targeted therapeutic carriers. Journal of Controlled Release, 2014. 196: p. 243-251.
Sadhukha, T.; Wiedmann, T. S.; Panyam, J., Inhalable magnetic nanoparticles for targeted hyperthermia in lung cancer therapy. Biomaterials 2013, 34 (21), 5163-71.
Sailor, M. J. and J. H. Park, Hybrid nanoparticles for detection and treatment of cancer. Advanced materials, 2012. 24(28): p. 3779-3802.
Schmid, D., et al., T cell-targeting nanoparticles focus delivery of immunotherapy to improve antitumor immunity. Nature Communications, 2017. 8(1): p. 1747.
Sciavolino, P. J., Autoimmunity and limitations of cancer vaccines. Targeted Oncology, 2014.
Scott, A. M., J. D. Wolchok, and L. J. Old, Antibody therapy of cancer. Nature Reviews Cancer, 2012. 12(4): p. 278.
Siegel, R.; Naishadham, D.; Jemal, A., Cancer statistics, 2013. CA: A Cancer Journal for Clinicians 2013, 63 (1), 11-30.
Simões, S., et al., Cationic liposomes for gene delivery. Expert opinion on drug delivery, 2005. 2(2): p. 237-254.
Singh, S., et al., Nanoparticle based drug delivery system: advantages and applications. Indian Journal of Science and Technology, 2011. 4(3): p. 177-180.
Socinski, M. A.; Rosenman, J. G., Chemotherapeutic issues in the management of unresectable stage III non-small cell lung cancer. Seminars in oncology 2005, 32 (2 Suppl 3), S18-24.
Spicer, J. D., et al., Neutrophils promote liver metastasis via Mac-1 mediated interactions with circulating tumor cells. Cancer Research, 2012: p. canres. 2393.2011.
Springer, T. A., Traffic signals for lymphocyte recirculation and leukocyte emigration: the multistep paradigm. Cell, 1994. 76(2): p. 301-314.
Stephan, M. T., et al., Synapse-directed delivery of immunomodulators using T-cell-conjugated nanoparticles. Biomaterials, 2012. 33(23): p. 5776-5787.
Strell, C., et al., Surface molecules regulating rolling and adhesion to endothelium of neutrophil granulocytes and MDA-MB-468 breast carcinoma cells and their interaction. Cellular and Molecular Life Sciences, 2007. 64(24): p. 3306-3316.
Sun, H., et al., Cancer cell membrane-coated gold nanocages with hyperthermia-triggered drug release and homotypic target inhibit growth and metastasis of breast cancer. Advanced Functional Materials, 2017. 27(3): p. 1604300.
Sun, W., et al., Endocytosis of a single mesoporous silica nanoparticle into a human lung cancer cell observed by differential interference contrast microscopy. Analytical and Bioanalytical Chemistry, 2008. 391(6): p. 2119.
Sundarraj, S., EGFR antibody conjugated mesoporous silica nanoparticles for cytosolic phospholipase A2α targeted nonsmall lung cancer therapy. Journal of Cell Science and Therapy, 2012. 3(7).
Tarazona, R., et al., Immunosenescence: limitations of natural killer cell-based cancer immunotherapy. Cancer Immunology, Immunotherapy, 2017. 66(2): p. 233-245.
Thamphiwatana, S., et al., Macrophage-like nanoparticles concurrently absorbing endotoxins and proinflammatory cytokines for sepsis management. Proceedings of the National Academy of Sciences, 2017. 114(43): p. 11488-11493.
Thanuja, M., C. Anupama, and S. H. Ranganath, Bioengineered cellular and cell membrane-derived vehicles for actively targeted drug delivery: So near and yet so far. Advanced Drug Delivery Reviews, 2018.
Thanuja, M., C. Anupama, and S. H. Ranganath, Bioengineered cellular and cell membrane-derived vehicles for actively targeted drug delivery: So near and yet so far. Advanced Drug Delivery Reviews, 2018. 132: p. 57-80.
Timmerman, R., et al., Excessive toxicity when treating central tumors in a phase II study of stereotactic body radiation therapy for medically inoperable early-stage lung cancer. Journal of Clinical Oncology, 2006. 24(30): p. 4833-4839.
Toledano Furman, N. E., et al., Reconstructed stem cell nanoghosts: a natural tumor targeting platform. Nano Letters, 2013. 13(7): p. 3248-3255.
Trinidad, A. J., et al., Combined concurrent photodynamic and gold nanoshell loaded macrophage-mediated photothermal therapies: An in vitro study on squamous cell head and neck carcinoma. Lasers in Surgery and Medicine, 2014. 46(4): p. 310-318.
Tsai, R. K., P. L. Rodriguez, and D. E. Discher, Self inhibition of phagocytosis: the affinity of ‘marker of self’ CD47 for SIRPα dictates potency of inhibition but only at low expression levels. Blood Cells, Molecules, and Diseases, 2010. 45(1): p. 67-74.
Valencia, P. M., et al., Effects of ligands with different water solubilities on self-assembly and properties of targeted nanoparticles. Biomaterials, 2011. 32(26): p. 6226-6233.
Von Maltzahn, G., et al., Nanoparticles that communicate in vivo to amplify tumour targeting. Nature Materials, 2011. 10(7): p. 545.
Von Roemeling, C., et al., Breaking down the barriers to precision cancer nanomedicine. Trends in Biotechnology, 2017. 35(2): p. 159-171.
Wang, D., et al., Erythrocyte-Cancer Hybrid Membrane Camouflaged Hollow Copper Sulfide Nanoparticles for Prolonged Circulation Life and Homotypic-Targeting Photothermal/Chemotherapy of Melanoma. ACS nano, 2018. 12(6): p. 5241-5252.
Wang, F., et al., Nanoparticle-based antivirulence vaccine for the management of methicillin-resistant Staphylococcus aureus skin infection. Advanced Functional Materials, 2016. 26(10): p. 1628-1635.
Wang, X., et al., Mesenchymal stem cells loaded with paclitaxel-poly (lactic-co-glycolic acid) nanoparticles for glioma-targeting therapy. International Journal of Nanomedicine, 2018. 13: p. 5231.
Wang, Y., et al., Detection of micrometastases in lung cancer with magnetic nanoparticles and quantum dots. International Journal of Nanomedicine, 2012. 7: p. 2315.
Warenius, H., et al., Attempted targeting of a monoclonal antibody in a human tumour xenograft system. European Journal of Cancer and Clinical Oncology, 1981. 17(9): p. 1009-1015.
Weidner, A., et al. Formation of a biocompatible protein corona on magnetic nanoparticles. In Magnetic Particle Imaging (IWMPI), 2015 5^{th} International Workshop on. 2015. IEEE.
Weiner, L. M., R. Surana, and S. Wang, Monoclonal antibodies: versatile platforms for cancer immunotherapy. Nature Reviews Immunology, 2010. 10(5): p. 317.
Wicki, A., et al., Nanomedicine in cancer therapy: challenges, opportunities, and clinical applications. Journal of Controlled Release, 2015. 200: p. 138-157.
Wu, H.-H., et al., Mesenchymal stem cell-based drug delivery strategy: from cells to biomimetic. Journal of Controlled Release, 2018.
Xin, H., et al., Angiopep-conjugated poly (ethylene glycol)-co-poly (ε-caprolactone) nanoparticles as dual-targeting drug delivery system for brain glioma. Biomaterials, 2011. 32(18): p. 4293-4305.
Xuan, M., et al., Nanocapsules: macrophage cell membrane camouflaged mesoporous silica nanocapsules for in vivo cancer therapy (Adv. Healthcare Mater. November 2015). Advanced Healthcare Materials, 2015. 4(11): p. 1578-1578.
Xue, J., et al., Neutrophil-mediated anticancer drug delivery for suppression of postoperative malignant glioma recurrence. Nature Nanotechnology, 2017. 12(7): p. 692.
Yaman, S.; Chintapula, U.; Rodriguez, E.; Ramachandramoorthy, H.; Nguyen, K. T., Cell-mediated and cell membrane-coated nanoparticles for drug delivery and cancer therapy. Cancer Drug Resistance 2020, 3 (4), 879-911.
Yaman, S.; Ramachandramoorthy, H.; Oter, G.; Zhukova, D.; Nguyen, T.; Sabnani, M. K.; Weidanz, J. A.; Nguyen, K. T., Melanoma Peptide MHC Specific TCR Expressing T-Cell Membrane Camouflaged PLGA Nanoparticles for Treatment of Melanoma Skin Cancer. Frontiers in Bioengineering and Biotechnology 2020, 8, 943.
Yin, H., et al., Reducing the cytotoxicity of ZnO nanoparticles by a pre-formed protein corona in a supplemented cell culture medium. RSC Advances, 2015. 5(90): p. 73963-73973.
Yoo, J.-W., et al., Bio-inspired, bioengineered and biomimetic drug delivery carriers. Nature Reviews Drug discovery, 2011. 10(7): p. 521.
Yu, X., et al., Design of nanoparticle-based carriers for targeted drug delivery. Journal of Nanomaterials, 2016. 2016.
Yumura, K., et al., Mutations for decreasing the immunogenicity and maintaining the function of core streptavidin. Protein Science, 2013. 22(2): p. 213-221.
Zanganeh, S., et al., Protein corona: Opportunities and challenges. The international journal of biochemistry & cell biology, 2016. 75: p. 143-147.
Zappa, C. and S. A. Mousa, Non-small cell lung cancer: current treatment and future advances. Translational lung cancer research, 2016. 5(3): p. 288.
Zhang, D., J. Wang, and D. Xu, Cell-penetrating peptides as noninvasive transmembrane vectors for the development of novel multifunctional drug-delivery systems. Journal of Controlled Release, 2016. 229: p. 130-139.
Zhang, J.; Tang, H.; Liu, Z.; Chen, B., Effects of major parameters of nanoparticles on their physical and chemical properties and recent application of nanodrug delivery system in targeted chemotherapy. International journal of nanomedicine 2017, 12, 8483-8493.
Zhang, L., et al., Human cytotoxic T-lymphocyte membrane-camouflaged nanoparticles combined with low-dose irradiation: a new approach to enhance drug targeting in gastric cancer. International Journal of Nanomedicine, 2017. 12: p. 2129.
Zhang, Q.; Shan, W.; Ai, C.; Chen, Z.; Zhou, T.; Lv, X.; Zhou, X.; Ye, S.; Ren, L.; Wang, X., Construction of Multifunctional Fe<sub>3</sub>O<sub>4</sub>-MTX@HBc Nanoparticles for MR Imaging and Photothermal Therapy/Chemotherapy. Nanotheranostics 2018, 2 (1), 87-95.
Zhang, Y., et al., Macrophage-membrane-coated nanoparticles for tumor-targeted chemotherapy. Nano Letters, 2018. 18(3): p. 1908-1915.
Zhong, Y., et al., Ligand-directed active tumor-targeting polymeric nanoparticles for cancer chemotherapy. Biomacromolecules, 2014. 15(6): p. 1955-1969.
Zhu, J.-Y., et al., Preferential cancer cell self-recognition and tumor self-targeting by coating nanoparticles with homotypic cancer cell membranes. Nano Letters, 2016. 16(9): p. 5895-5901.

1. A chimeric antigen receptor (CAR) T cell membrane coated particle (CAR-T-MNP) comprising i) a T cell membrane comprising a CAR on its cell membrane and ii) an engineered particle; wherein the engineered particle comprises a therapeutic agent.

2. The CAR-T-MNP of claim 1, wherein the CAR comprises a first specificity specific for a cancer cell specific marker.

3. The CAR-T-MNPs of claim 1, wherein the CAR-T-MNP comprises a i) bi-specific CAR comprising a CAR-T plasma membrane with an intracellular facing binding moiety and an extracellular facing binding moiety and/or ii) two or more differently targeting CARs.

4. The CAR-T-MNPs of claim 3, an extracellular binding moiety, a hinge region, a transmembrane domain, an intracellular hinge region, and an intracellular binding moiety. In one aspect, the intracellular binding moiety and extracellular binding moiety.

5. The CAR-T-MNPs of claim 1, wherein the first and second specificity are to the same target.

6. The CAR-T-MNP of claim 1, wherein the engineered particle comprises poly(lactic co-glycolic) acid (PLGA).

7. The CAR-T-MNP of claim 1, wherein the CAR is specific for carcinoembryonic antigen (CEA), EGFRVIII, IL-llRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin Bl, lectin-reactive AFP, Fos-related antigen 1, ADRB3, thyroglobulin, EphA2, RAGE-1, Rul, RU2, SSX2, AKAP-4, LCK, OY-TESl, PAX5, SART3, CLL-1, fucosyl GM1, GloboH, MN-CA IX, EPCAM, EVT6-AML, TGS5, human telomerase reverse transcriptase, plysialic acid, PLAC1, Rul, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (Fra), folate receptor beta, ROR1, Flt3, TAG72, TN Ag, Tie 2, TEM1, TEM7R, CLDN6, TSHR, UPK2, and/or mesothelin.

8. The CAR-T-MNP of claim 1, wherein the therapeutic agent comprises a small molecule, RNAi, gene vector, peptide, polypeptide, protein, or antibody.

9. The CAR-T-MNP of claim 1, wherein the therapeutic agent comprises an anticancer agent.

10. The CAR-T-MNP of claim 1, wherein the therapeutic agent comprises an antimicrobial agent.

11. The CAR-T-MNP of claim 1, wherein the therapeutic agent comprises an anti-inflammatory agent.

12. A method of targeting the delivery of a therapeutic agent to a target site in a subject in need thereof comprising administering to the subject any of the CAR-T-MNP of claim 1.

13. A method of treating, inhibiting, reducing, ameliorating, and/or preventing a cancer and/or metastasis in a subject comprising administering to the subject any of the CAR-T-MNP of claim 1.

14. The method of treating, inhibiting, reducing, ameliorating, and/or preventing a cancer and/or metastasis of claim 13, wherein the cancer comprises non-small cell lung cancer or breast cancer.

15. A method of treating, inhibiting, reducing, ameliorating, and/or preventing a microbial infection in a subject comprising administering to the subject any of the CAR-T-MNP of claim 1.

16. The method of treating, inhibiting, reducing, ameliorating, and/or preventing a microbial infection of claim 15, wherein the microbial infection is a viral infection.

17. (canceled)

18. The method of treating, inhibiting, reducing, ameliorating, and/or preventing a microbial infection of claim 16, wherein the antimicrobial agent comprises an antiviral agent selected from the group consisting of acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; levetiracetam; tramadol; or any of their isomers tautomers, analogs, polymorphs, solvates, derivatives, or pharmaceutically acceptable salts.

19. The method of treating, inhibiting, reducing, ameliorating, and/or preventing a microbial infection of claim 15, wherein microbial infection is a bacterial infection.

20. (canceled)

21. The method of treating, inhibiting, reducing, ameliorating, and/or preventing a microbial infection of claim 19, wherein the antimicrobial agent comprises an antibiotic agent selected from the group consisting of amikacin, gentamicin, kanamycin, neomycin, tobramycin, paramomycin, streptomycin, spectinomycin, geldanamycin, herbimycin, rifaximin, loracarbef, ertapenem, doripenem, imipenem, meropenem, cefadroxil, cefazolin, cephradine, cephapirin, cephalothin, cefalexin, cefprozil, loracarbef, cefonicid, cemetazole, cefamandole, cefdinir, cefoperazone, cefotaxime, cefpodoxime, ceftazidime, ceftabiprole, Ceftaroline fosamil, vancomycin, dalbavancin, lincomycin, cleocin, oritavancin, dalbavancin, daptomycin, spriamycin, linezolid, penicillin, amoxicillin, ampicillin, dicloxacillin, methicillin, oxacillin, bacitracin, colistin, polymyxin B, ciprofloxacin, enoxacin, Gemifloxacin, levofloxacin, ofloxacin, mefanide, sulfacetamide, sulfadiazine, sulfamethizole, doxycycline, tetracycline, oxytertracycline, cycloserine, pyrazinamide, rifampicin, isoniazid, dapsone, clofazimine, pyrszinamide, or any of their isomers tautomers, analogs, polymorphs, solvates, derivatives, or pharmaceutically acceptable salts.

22. The method of treating, inhibiting, reducing, ameliorating, and/or preventing a microbial infection of claim 15, wherein the microbial infection is a fungal infection.

23. (canceled)

24. The method of treating, inhibiting, reducing, ameliorating, and/or preventing a microbial infection of claim 22, wherein the antimicrobial agent comprises an antifungal agent selected from the group consisting of bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, propiconazole, ravuconazole, terconazole, or any of their isomers tautomers, analogs, polymorphs, solvates, derivatives, or pharmaceutically acceptable salts.

25. A method of treating, inhibiting, reducing, ameliorating, and/or preventing an inflammatory condition in a subject comprising administering to the subject any of the CAR-T-MNP of claim 1.
